(19) United States
(12) Patent Application Publication
(10) Pub. No.: US 2014/0107351 A1
(43) Pub. Date: Apr. 17, 2014

(54) Fullerene-Containing Hemicarceplexes and a Method of Purifying Fullerenes by Using the Same

(71) Applicant: NATIONAL TAIWAN UNIVERSITY, Taipei (TW)

(72) Inventors: Sheng-Hsien Chiu, Taipei City (TW); Ming-Jhe Li, Ji'an Township (TW)

(73) Assignee: NATIONAL TAIWAN UNIVERSITY, Taipei (TW)

(21) Appl. No.: 13/653,539

(22) Filed: Oct. 17, 2012

(51) Int. Cl. C07D 493/22

(52) U.S. Cl. 549/354

(57) ABSTRACT

Fullerene⊙CTV complexes, comprising fullerene⊙CTV hemicarceplexes, formed by various cyclotriveratrylene (CTV)-based molecular cages and various fullerene guests are disclosed. A method of direct isolating at least a fullerene from fullerene mixtures by using the above fullerene CTV hemicarceplexes but without using crystallization or HPLC is also disclosed.

### BACKGROUND

1. Technical Field

The disclosure relates to several fullerene-containing hemicarceplexes and a method of purifying fullerenes by using the same.

2. Description of Related Art

Because of their versatile configurations and attractive properties, fullerenes, including cylindrical carbon nanotubes (CNTs) and spherical and spheroidal buckyballs, have found applications in a wide range of fields, including materials science, chemistry, super- and semi-conducting physics, and biology. Ignoring CNTs, which lack uniform diameters or lengths, the most abundant structurally distinct species in a typical fullerene extract are two buckyballs, i.e. C₆₀ and C₇₀. Even though they have been investigated widely since their discovery in the 1980s, the practical applications of buckyballs have been limited by their poor solubilities in organic solvents; this characteristic has also seriously complicated their isolation and purification.

Several elegant methods have been developed for the isolation of the more-abundant C₆₀ from fullerene extracts; in contrast, isolating the lower-in-symmetry and photovoltaically-more-interesting C₇₀ in high purity from the same mixtures has been less straightforward. Indeed, tedious purification involving crystallization and/or high-performance liquid chromatography (HPLC) is frequently required to obtain high-purity C₇₀, making it much less affordable than C₆₀ of the same quality; accordingly, relatively limited research has been undertaken to discover and expand the practical applications of C₇₀.

One attractive approach for the selective isolation of C₇₀ involves exploiting its host-guest complexation behavior. Although a few judiciously designed synthetic host molecules do form complexes with C₆₀ and C₇₀ in solution, using such host-guest complexes as a means of separating mixtures of buckyballs (i.e., with high degrees of selectivity and stability) remains a challenge.

Unlike carcerands, which cannot release their entrapped guests, hemicarcerands allow sequestration of complementary guests (forming room temperature-isolatable hemicarceplexes) as well as their release at elevated temperatures. Although Cram first proposed, in 1995, that the internal space of a cavitand dimer might be a suitable host for C₆₀ (Hemicarcerands with interiors potentially capable of binding large guests. J. Chem. Soc., Chem. Commun. 1085-1087 (1995)), hemicarcerands that can selectively imprison guests as big as C₆₀ and C₇₀ have never been realized previously, presumably because of difficulties in balancing the steric sizes and free energies of complexation of the host and guest components to allow selective sequestration and release of the guests.

### SUMMARY

In one aspect, the present invention is directed to a fullerene⊙CTV complex, comprising a hemicarceplex, formed by trapping a fullerene guest or a derivative thereof in a cyclotriveratrylene-based molecular cage (abbreviated as CTV below) having a chemical structure below, and LS1 and LS2 are first and second linking spacers.

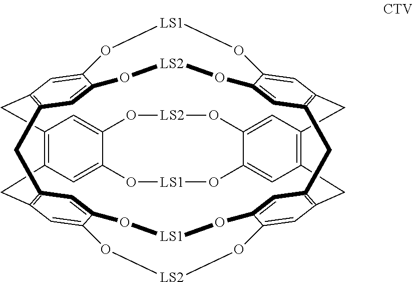

According to an embodiment, at least three of the first and the second linking spacers are alkyl chains containing at least 10 carbons. For example, the cyclotriveratrylene-based molecular cage cane be

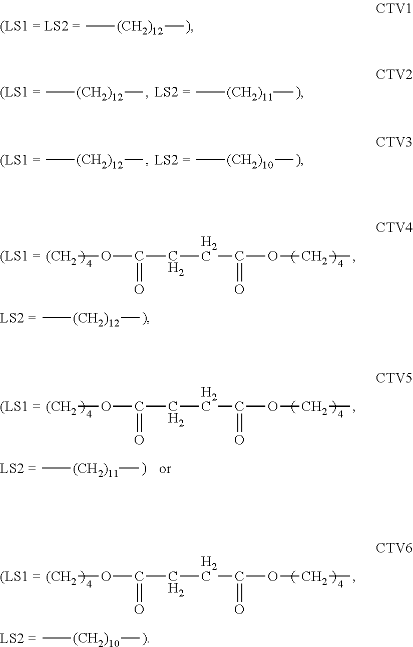

According to another embodiment, at least one of the first and the second linking spacers containing a diester linkage. For example, the cyclotriveratrylene-based molecular cage can be

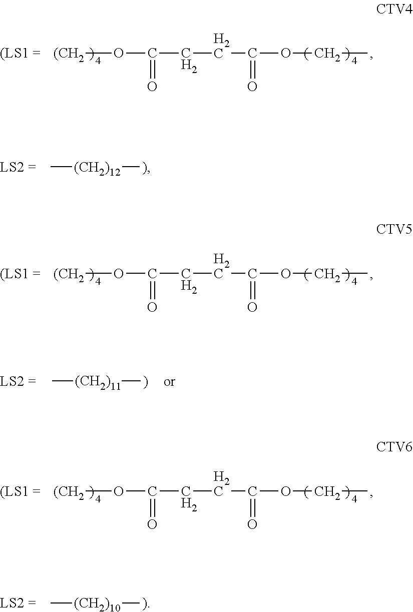

According to yet another embodiment, the complex can be C₆₀⊙CTV1, C₇₀⊙CTV1, C₇₆⊙CTV1, C₇₈⊙CTV1, C₇₀⊙CTV2, C₆₀⊙CTV2, C₆₀⊙CTV3, Sc₃N@C₈₀⊙CTV4, C₆₀⊙CTV5, C₇₀⊙CTV5, C₇₆⊙CTV5 or C₇₈⊙CTV5, or C₆₀ ⊙CTV6.

According to yet another embodiment, the complex can be C₇₀⊙CTV1, C₇₆⊙CTV1, C₇₈⊙CTV1, C₇₀⊙CTV2, C₆₀⊙CTV3, Sc₃N@C₈₀⊙CTV4, or C₇₆⊙CTV5 or C₇₈⊙CTV5 when the complex is room temperature isolatable.

In another aspect, the present invention directs to a method of forming a fullerene⊙CTV hemicarceplex. The method comprises the following steps. A fullerene or a derivative thereof, and a cyclotriveratrylene-based molecular cage described above are mixed in a solvent to form a mixture solution. Then, the mixture solution is heated to form a fullerene⊙CTV hemicarceplex.

According to an embodiment, the solvent can majorly contain CS₂, CH₂Cl₂, CHCl₃ or CHCl₂CHCl₂, for example.

In yet another aspect, the present invention directs to a method of isolating at least a fullerene by using a fullerene⊙CTV hemicarceplex. The method comprises the following steps. First, a fullerene or a derivative thereof, and a cyclotriveratrylene-based molecular cage described above are mixed in a first solvent to form a mixture solution. Next, the fullerene⊙CTV hemicarceplexe is isolated by column chromatography without using crystallization or high performance liquid chromatography (HPLC). Then, the fullerene⊙CTV hemicarceplexe is dissociated in a second solvent.

According to an embodiment, the first solvent has less tendency than the fullerenes to occupy an inner space of the cyclotriveratrylene-based molecular cage. For example, the first solvent can majorly contain CS₂, CH₂Cl₂, CHCl₃, or CHCl₂CHCl₂.

According to another embodiment, the second solvent can dissolve fullerene⊙CTV hemicarceplex and allow its dissociation to release fullerene. For example, the second solvent can majorly contain CS₂, CH₂Cl₂, CHCl₃, CHCl₂CHCl₂, benzene, toluene, or dichlorobenzene.

The forgoing presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later. Many of the attendant features will be more readily appreciated as the same becomes better understood by reference to the following detailed description considered in connection with the accompanying drawings.

### BRIEF DESCRIPTION OF THE DRAWINGS

FIGS. 1A and 1B are ¹H NMR (400 MHz, CDCl₃, 298 K) and ¹³C NMR (100 MHz, CDCl₃, 298 K) spectra of CTV1, respectively.

FIGS. 2A and 2B are ¹H NMR (400 MHz, CDCl₃, 298 K) and ¹³C NMR (100 MHz, CDCl₃, 298 K) spectra of CTV2, respectively.

FIGS. 3A and 3B are ¹H NMR (400 MHz, CDCl₃, 298 K) and ¹³C NMR (100 MHz, CDCl₃, 298 K) spectra of CTV3, respectively.

FIGS. 4A and 4B are ¹H NMR (400 MHz, CDCl₃, 298 K) and ¹³C NMR (100 MHz, CDCl₃, 298 K) spectra of CTV4, respectively.

FIGS. 5A and 5B are ¹H NMR (400 MHz, CDCl₃, 298 K) and ¹³C NMR (100 MHz, CDCl₃, 298 K) spectra of CTV5, respectively.

FIGS. 6A and 6B are ¹H NMR (400 MHz, CDCl₃, 298 K) and ¹³C NMR (100 MHz, CDCl₃, 298 K) spectra of CTV6, respectively.

FIGS. 7A, 7B, 7C, and 7D are ¹H NMR spectrum (400 MHz, CDCl₂CDCl₂, 298 K) of CTV1, an equimolar mixture of C₆₀ and CTV1, an equimolar mixture of C₇₀ and CTV1, and purified C₇₀ CTV1 hemicarceplex, respectively.

FIGS. 8A, 8B, 8C, and 8D are ¹³C NMR spectrum (400 MHz, CDCl₂CDCl₂, 298 K) of CTV1, an equimolar mixture of C₆₀ and CTV1, purified C₇₀, and purified C₇₀ CTV1 hemicarceplex, respectively.

FIGS. 9A and 9B are ¹H NMR (400 MHz, CDCl₃, 298 K) and ¹³C NMR (100 MHz, CDCl₃, 298 K) spectra of hemicarceplex C₇₀ CTV2, respectively.

FIGS. 10A and 10B are ¹H NMR (400 MHz, CDCl₃, 298 K) and ¹³C NMR (200 MHz, CDCl₃, 298 K) spectra of hemicarceplex C₆₀ CTV3, respectively.

FIG. 11 is the ¹H NMR (400 MHz, CDCl₃, 298 K) spectrum of hemicarceplex Sc3@C₈₀ CTV4.

FIGS. 12A and 12B are ¹H NMR (400 MHz, CDCl₃, 298 K) spectra of the equimolar mixture of CTV5 to C₆₀ and C₇₀, respectively.

FIGS. 13A and 13B are the ¹H NMR (400 MHz, CDCl₃, 298 K) spectra of free CTV6, and the equimolar mixture of CTV6 and C₆₀, respectively.

FIG. 14 is a process flow diagram of isolating fullerene⊙CTV hemicarceplexes by column chromatography.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details.

### Synthesizing Cyclotriveratrylenes (CTVs) for Forming Fullerene CTV Hemicarceplexes

Cyclotriveratrylene-based molecular cages (abbreviated as CTVs below) for forming fullerene CTV complexes or hemicarceplexes were synthesized first. The CTV host molecule has a chemical structure shown below, wherein LS1 and LS2 represent first and second linking spacers.

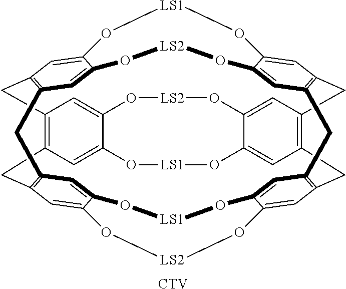

According to an embodiment, at least three of the first and second linking spacers are alkyl chains containing at least 10 carbons, such as 10-15 carbons. According to another embodiment, at least one of the first and second linking spacers containing a diester linkage. Six CTV host molecules were synthesized, and the first and the second linking spacers are listed in the table below.

| CTV | | |
|---|---|---|
| host | LS1 | LS2 |
| CTV1 | —(CH₂)₁₂— | —(CH₂)₁₂— |
| CTV2 | —(CH₂)₁₂— | —(CH₂)₁₁— |
| CTV3 | —(CH₂)₁₂— | —(CH₂)₁₀— |
| CTV4 | 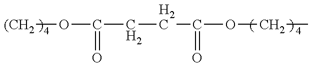 | —(CH₂)₁₂— |
| CTV5 | 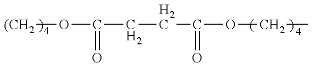 | —(CH₂)₁₁— |
| CTV6 | 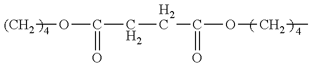 | —(CH₂)₁₀— |

### Synthesis of CTV1

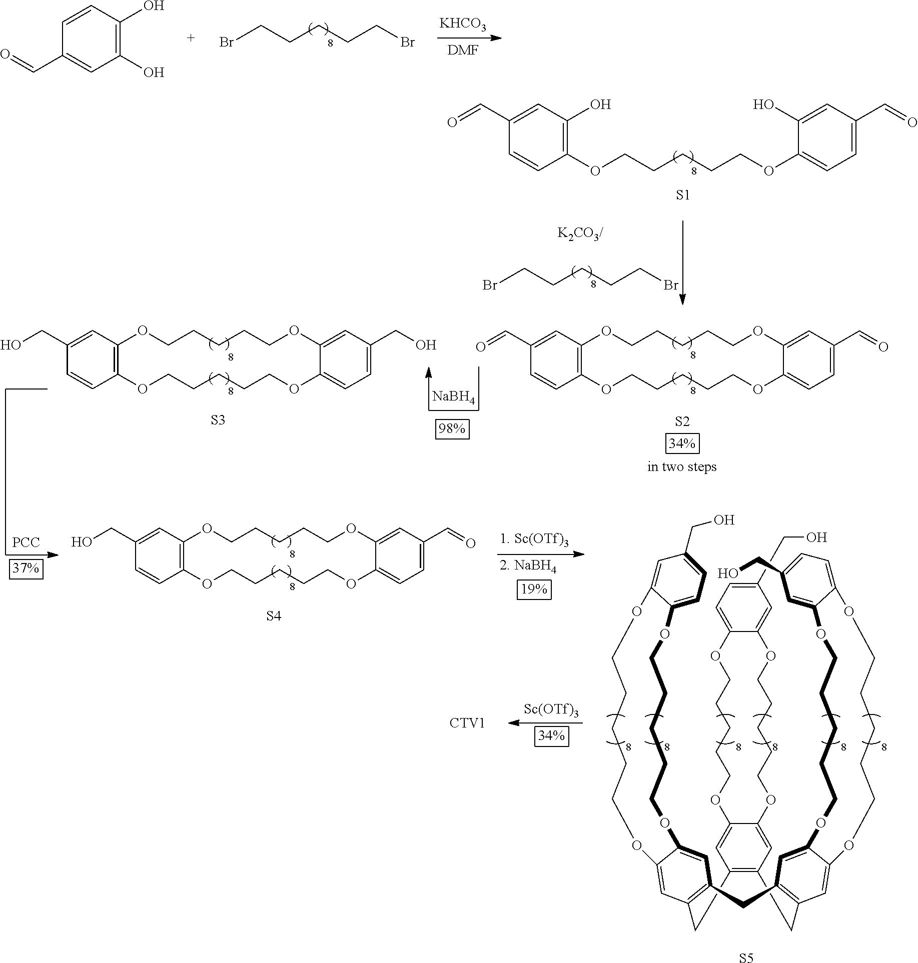

Dialdehyde S2:

The reaction of 3,4-dihydroxybenzaldehyde (5.17 g, 37.4 mmol), 1,12-dibromododecane (5.58 g, 17.0 mmol), and KHCO₃ (3.74 g, 37.4 mmol) in DMF (75 mL) at 65° C. for 3 days afforded the monoalkylated-dialdehyde S1, which was dissolved with 1,12-dibromodecane (3.71 g, 11.3 mmol) in DMF (130 mL) and reacted with K₂CO₃ (9.37 g, 67.8 mmol) in DMF (1 L) to afford a white solid S2 (2.35 g, 34%).

Mp: 175-176° C.; ¹H NMR (400 MHz, CDCl₃, 298 K): δ=1.25-1.39 (m, 24H), 1.45-1.55 (m, 8H), 1.75-1.86 (m, 8H), 4.03 (t, J=5.6 Hz, 4H), 4.06 (t, J=105.6 Hz, 4H), 6.92 (d, J=8 Hz, 2H), 7.37 (d, J=2 Hz, 2H), 7.40 (dd, J=8, 2 Hz, 2H), 9.81 (s, 2H); ¹³C NMR (100 MHz, CDCl₃, 298 K): δ=26.4, 26.4, 29.2, 29.3, 29.6, 29.6, 29.8, 29.8, 29.8, 29.8, 69.0, 69.1, 111.2, 111.9, 126.6, 129.9, 149.6, 154.9, 191.0; HR-MS (ESI): calcd for C₃₈H₅₆O₆Na⁺ [M+Na]⁺, m/z 631.3975. found, m/z 631.3972.

Diol S3:

Following the procedure described above for S2, the reaction of the dialdehyde S2 (1.92 g, 3.16 mmol) and NaBH₄ (0.36 g, 9.47 mmol) in isopropyl alcohol (79 mL) and CH₂Cl₂ (79 mL) under reflux for 16 h afforded a white solid S3 (1.89 g, 98%).

Mp: 153-154° C.; ¹H NMR (400 MHz, CDCl₃, 298 K): δ=1.18-1.38 (m, 24H), 1.43-1.53 (m, 8H), 1.73-1.82 (m, 8H), 3.97 (t, J=6.4 Hz, 4H), 3.98 (t, J=6.4 Hz, 4H), 4.58 (s, 4H), 6.84 (s, 4H), 6.91 (s, 2H); ¹³C NMR (100 MHz, CDCl₃, 298 K): δ=26.1, 26.3, 26.4, 29.4, 29.5, 29.5, 29.6, 29.7, 29.8, 29.8, 65.4, 69.3, 69.5, 113.2, 114.2, 119.7, 133.8, 149.0, 149.6; HR-MS (ESI): calcd for C₃₈H₆₀O₆Na⁺ [M+Na]⁺, m/z 635.4288. found, m/z 635.4285.

Mono-Alcohol S4:

Following the procedure described above for S3, the reaction of the diol S3 (0.1 g, 0.163 mmol), pyridinium chlorochromate (53 mg, 245 μmole), 4-Å molecular sieves (0.75 g), and Celite (1.49 g) in CH₂Cl₂ (5.2 mL) and DMF (3 mL) at 60° C. for 3 h afforded a white solid S4 (39 mg, 37%).

Mp: 166-168° C.; ¹H NMR (400 MHz, CDCl₃, 298 K): δ=1.25-1.39 (m, 24H), 1.44-1.55 (m, 8H), 1.73-1.86 (m, 8H), 3.93-4.01 (m, 4H), 4.01-4.09 (m, 4H), 4.58 (d, J=5.2 Hz, 2H), 6.84 (s, 2H), 6.91-6.93 (m, 2H), 7.36-7.42 (m, 2H), 9.81 (s, 1H); ¹³C NMR (100 MHz, CDCl₃, 298 K): δ=26.4, 29.2, 29.3, 29.6, 29.6, 29.7, 29.8, 29.8, 65.4, 69.1, 69.1, 69.3, 69.5 (12 signals are missing, possibly because of signal overlapping), 111.2, 111.9, 113.2, 114.2, 119.7, 126.6, 129.9, 133.9, 148.9, 149.6, 149.6, 154.9, 191.0; HR-MS (ESI): calcd for C₃₈H₅₈O₆Na⁺ [M+Na]⁺, m/z 633.4131. found, m/z 633.4141.

Triol S5:

Following the procedure described above for S4, the reaction of the mono-alcohol S4 (1.33 g, 2.18 mmol) and Sc(OTf)₃ (54 mg, 0.11 mmol) in CHCl₃ (11 mL) at 70° C. for 16 h afforded the trialdehyde as a light yellow solid, which was reacted with NaBH₄ (50 mg, 1.21 mmole) in isopropyl alcohol (30 mL) and CH₂Cl₂ (30 mL) at room temperature for 16 h to afford a white solid S5 (0.25 g, 19%).

Mp: 117-119° C.; ¹H NMR (400 MHz, CDCl₃, 298 K): δ=1.20-1.52 (m, 96H), 1.67-1.81 (m, 24H), 3.46 (d, J=13.6 Hz, 3H), 3.82-3.89 (m, 6H), 3.92-3.99 (m, 18H), 4.56 (s, 6H), 4.68 (d, J=13.6 Hz, 3H), 6.80 (s, 6H), 6.83 (s, 6H), 6.89 (s, 3H); ¹³C NMR (100 MHz, CDCl₃, 298 K): δ=26.3, 26.3, 26.4, 29.5, 29.6, 29.6, 29.8, 29.8, 29.8, 36.4, 65.3, 69.2, 69.5, 69.7, 113.2, 114.2, 116.2, 119.7, 132.3, 133.9, 148.0, 148.9, 149.6 (12 aliphatic and 3 aromatic signals are missing, possibly because of signal overlapping); HR-MS (ESI): calcd for C₁₁₄H₁₇₄O₁₅⁺ [M]⁺, m/z 1783.2853. found, m/z 1783.2791.

CTV1:

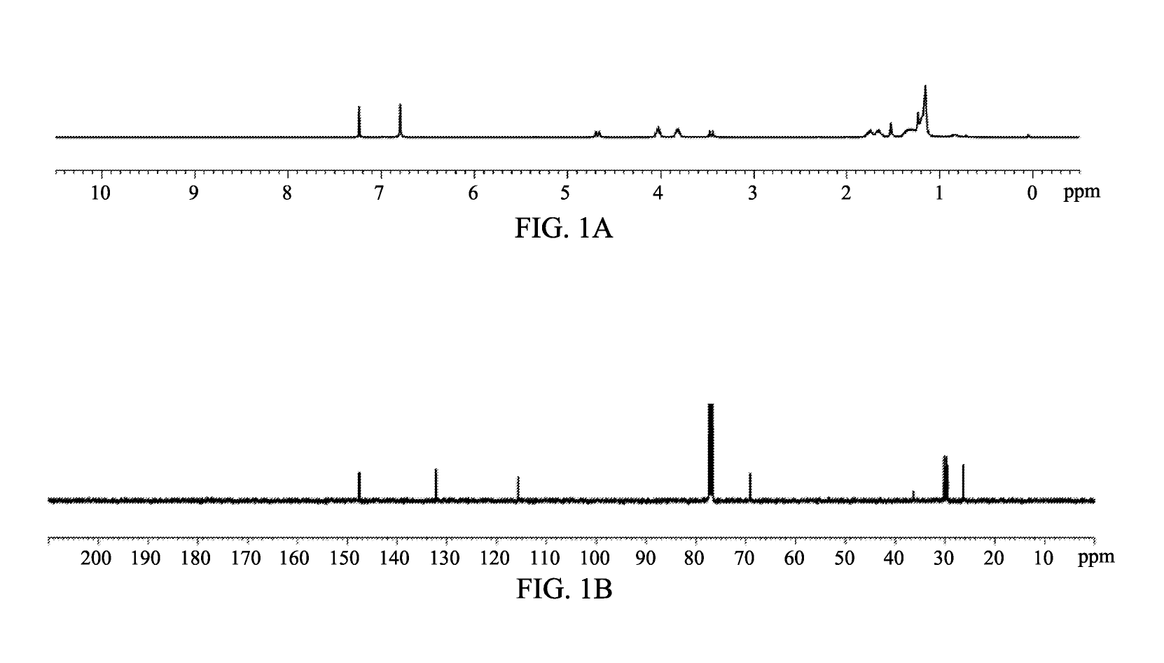

Following the procedure described above for S5, the reaction of the triol S5 (0.10 g, 0.056 mmol) and scandium triflate (60 mg, 0.12 mmol) in CHCl₃ (25 mL/30 mL) at 60° C. for 2 days afforded a white solid CTV1 (33 mg, 34%). The ¹H NMR and ¹³C NMR spectra of CTV1 are shown in FIGS. 1A and 1B. All related spectral data are listed below.

Mp: 258° C. (dec); ¹H NMR (400 MHz, CDCl₃, 298 K): δ=1.12-1.42 (m, 96H), 1.60-1.83 (m, 24H), 3.46 (d, J=13.6 Hz, 6H), 3.77-3.86 (m, 12H), 4.00-4.07 (m, 12H), 4.68 (d, J=13.6, 6H), 6.80 (s, 12H); ¹³C NMR (100 MHz, CDCl₃, 298 K): δ=26.3, 29.4, 29.8, 30.2, 30.2, 36.4, 69.1, 115.6, 132.1, 147.6; HR-MS (ESI): calcd for C₁₁₄H₁₆₈O₁₂Na⁺ [M+Na]⁺, m/z 1752.2432. found, m/z 1752.2488.

### Synthesis of CTV2

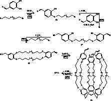

Aldehyde S6:

The reaction of potassium bicarbonate (0.67 g, 6.70 mmol), 3,4-dihydroxybenzaldehyde (0.93 g, 6.70 mmol), and 1,12-dibromododecane (2.00 g, 6.09 mmol) in DMF (60 mL) at 55° C. for 40 h afforded aldehyde S6 as a white solid (0.81 g, 34%).

Mp: 74-75° C.; ¹H NMR (400 MHz, CDCl₃, 298 K): δ=1.20-1.46 (m, 16H), 1.75-1.84 (m, 4H), 3.34 (t, J=6.8 Hz, 2H), 4.07 (t, J=6.6 Hz, 2H), 6.04 (s, 1H), 6.89 (d, J=8.2 Hz, 1H), 7.35 (dd, J=8.2, 2.0 Hz, 1H), 7.38 (d, J=2 Hz, 1H), 9.77 (s, 1H); ¹³C NMR (100 MHz, CDCl₃, 298 K): δ=25.8, 28.0, 28.6, 28.8, 29.1, 29.2, 29.3, 32.7, 33.9, 69.2 (2 signals are missing, possibly because of signal overlapping), 110.8, 113.9, 124.4, 130.2, 146.1, 151.3, 190.9; HR-MS (ESI): calcd for C₁₉H₃₀BrO₃⁺ [M+H]⁺, m/z 385.1373. found, m/z 385.1380

Alcohol S7:

Following the procedure described above for S6, the reaction of the aldehyde S6 (0.81 g, 2.10 mmol) and NaBH₄ (40 mg, 1.05 mmol) in methanol (200 mL) at room temperature for 2 h afforded a white solid (0.79 g, 97%). The white solid was then reacted with 3,4-dihydroxybenzaldehyde (0.31 g, 2.24 mmol) and potassium bicarbonate (0.23 g, 2.25 mmol) in DMF DMF (50 mL) at 55° C. for 36 h to afford a white solid S7 (0.58 g, 64%).

Mp: 136-137° C.; ¹H NMR (400 MHz, CDCl₃, 298 K): δ=1.25-1.50 (m, 16H), 1.73-1.89 (m, 4H), 4.01 (t, J=6.4 Hz, 2H), 4.11 (t, J=6.7 Hz, 2H), 4.56 (s, 2H), 5.67 (br, 2H), 6.80 (s, 2H), 6.90-6.96 (m, 2H), 7.36-7.44 (m, 2H), 9.81 (s, 1H); ¹³C NMR (100 MHz, CDCl₃, 298 K): δ=25.9, 26.0, 29.0, 29.2, 29.2, 29.3, 29.5, 29.5, 65.1, 69.1, 69.3 (2 signals are missing, possibly because of signal overlapping), 110.9, 111.6, 113.5, 114.1, 118.8, 124.4, 130.5, 134.2, 145.5, 145.9, 146.2, 151.3, 191.0; HR-MS (ESI): calcd for C₂₆H₃₅O₆⁻ [M−H]⁻, m/z 443.2439. found, m/z 443.2445.

Macrocycle S8:

Following the procedure described above for S7, the reaction of the diol S7 (3.74 g, 8.4 mmol), 1,11-dibromoundecane (2.6 4 g, 8.4 mmol) and K₂CO₃ (13.9 g, 101 mmol) in DMF (840 mL) at 60° C. for 5 days afforded a white solid S8 (2.01 g, 40%).

¹H NMR (400 MHz, CDCl₃, 298 K): δ=1.25-1.40 (m, 22H), 1.44-1.57 (m, 8H), 1.74-1.87 (m, 8H), 3.94-3.99 (m, 4H), 4.00-4.09 (m, 4H), 4.58 (s, 2H), 6.83 (s, 2H), 6.88-6.94 (m, 2H), 7.34-7.41 (m, 2H), 9.78 (s, 1H); ¹³C NMR (100 MHz, CDCl₃, 298 K): ¹³C NMR (100 MHz, CDCl₃, 298 K): δ=26.4, 26.4, 26.5, 26.6, 29.3, 29.4, 29.6, 29.6, 29.6, 29.7, 29.7, 29.8, 29.8, 29.8, 29.9, 30.0, 30.1, 65.3, 69.0, 69.1, 69.4, (3 signals missing, possibly because of signal overlap), 111.8, 111.6, 112.8, 113.9, 119.4, 126.5, 129.7, 133.7, 148.6, 149.3, 149.3, 154.6, 190.7; HR-MS (ESI): calcd for C₃₇H₅₆O₆Na⁺ [M+Na]⁺, m/z 619.40. found, m/z 619.39746.

Trialdehyde S9:

Following the procedure described above for S8, the reaction of the macrocycle S8 (0.2 g, 0.34 mmol) and Sc(OTf)₃ (8.4 mg, 0.017 mmol) in CHCl₃ (3.35 mL) at 70° C. for 16 h afforded a light-yellow solid S9 (55 mg, 29%).

¹H NMR (400 MHz, CDCl₃, 298 K): δ=1.20-1.55 (m, 90H), 1.68-1.87 (m, 24H), 3.46 (d, J=14.0 Hz, 3H), 3.82-4.10 (m, 24H), 4.68 (d, J=14.0 Hz, 3H), 6.80-6.83 (m, 6H), 6.89-6.94 (m, 3H), 7.34-7.41 (m, 6H), 9.79 (s, 3H); ¹³C NMR (100 MHz, CDCl₃, 298 K): δ=29.2, 29.3, 29.5, 29.6, 29.7, 29.7, 29.7, 29.9, 29.9, 29.9, 36.5, 69.1, 69.1, 69.5, 69.7 (9 signals are missing, possibly because of signal overlapping), 111.0, 111.8, 116.0, 116.3, 126.6, 129.9, 132.2, 132.3, 147.9, 148.0, 149.6, 154.8, 191.0; HR-MS (ESI): calcd for C₁₁₁H₁₆₂O₁₅Na⁺ [M+Na]⁺, m/z 1758.1811. found, m/z 1758.1812.

CTV2:

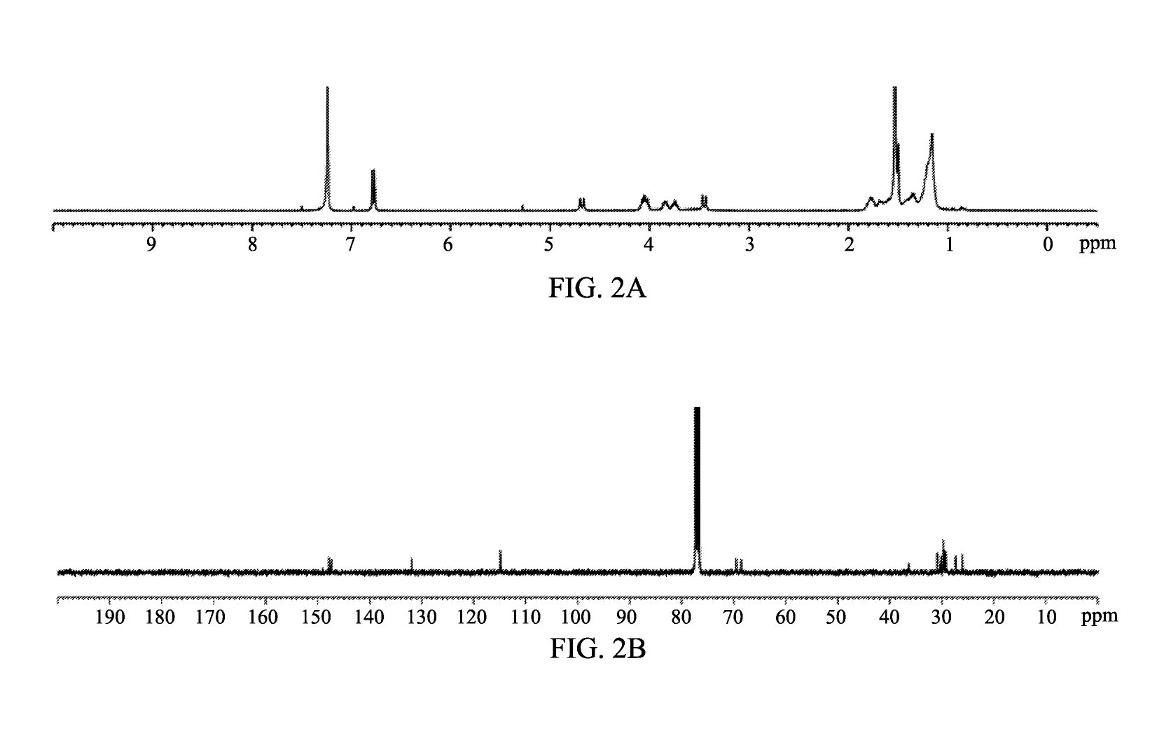

Following the procedure described above for S9, the reaction of the trialdehyde S9 (55 mg, 32 μmol) and NaBH₄ (4.84 mg, 0.13 mmol) in isopropyl alcohol (1 mL) and CH₂Cl₂ (1 mL) at room temperature for 16 h afford a white solid. The white solid was ten reacted with scandium triflate (11 mg, 23 μmol) in CHCl₃ (10 mL) at 60° C. for 3 days to afford a white solid CTV2 (15 mg, 28%). The ¹H NMR and ¹³C NMR spectra of CTV2 are shown in FIGS. 2A and 2B. All related spectral data are listed below.

Mp: >261° C. (dec.); ¹H NMR (400 MHz, CDCl₃, 298 K): δ=1.11-1.46 (m, 90H), 1.57-1.85 (m, 24H), 3.45 (d, J=13.8 Hz, 6H), 3.71-3.88 (m, 12H), 4.00-4.10 (m, 12H), 4.68 (d, J=13.7, 6H), 6.77 (s, 6H), 6.79 (s, 6H); ¹³C NMR (100 MHz, CDCl₃, 298 K): δ=26.0, 27.3, 29.2, 29.3, 29.6, 29.7, 30.1, 30.3, 30.8, 36.3, 68.5, 69.5, 114.9, 131.9, 147.4, 147.8 (3 signals missing, possibly because of signal overlap); HR-MS (ESI): calcd for C₁₁₁H₁₆₂O₁₂Na⁺ [M+Na]⁺, m/z 1710.20. found, m/z 1710.19641.

### Synthesis of CTV3

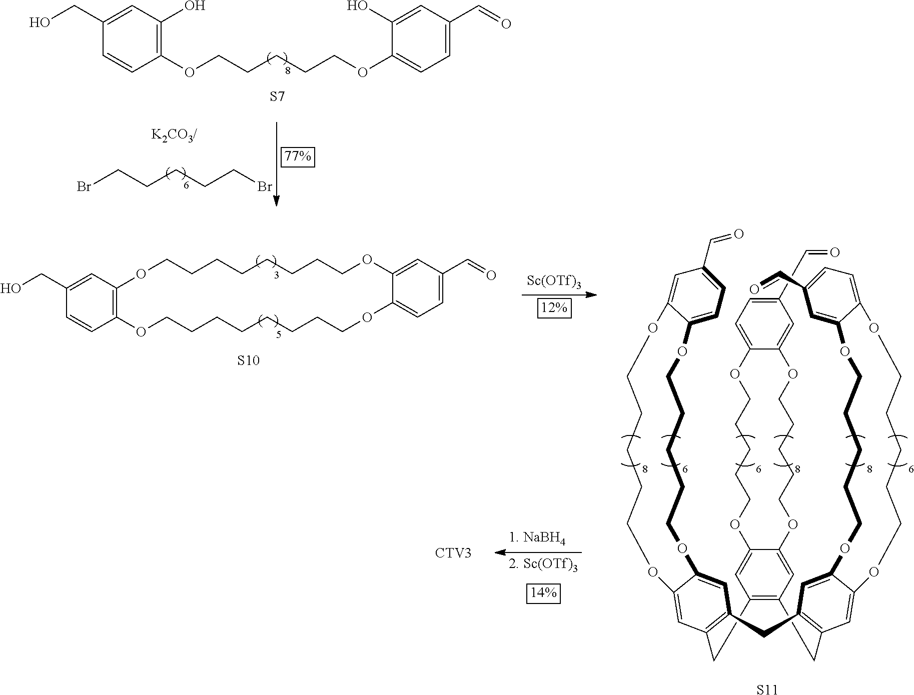

Macrocycle S10:

Following the procedure described above for S7, the reaction of the diol S7 (3.74 g, 8.4 mmol), 1,10-dibromodecane (2.52 g, 8.4 mmol) and K₂CO₃ (13.9 g, 101 mmol) in DMF (840 mL) at 60° C. for 5 days afforded a white solid S10 (1.88 g, 77%).

Macrocycle S10: ¹H NMR (400 MHz, CDCl₃, 298 K): δ=1.26-1.40 (m, 20H), 1.45-1.53 (m, 8H), 1.73-1.86 (m, 8H), 3.95-4.00 (m, 4H), 4.01-4.09 (m, 4H), 4.58 (s, 2H), 6.83 (s, 2H), 6.89-6.94 (m, 2H), 7.36-7.41 (m, 2H), 9.80 (s, 1H); ¹³C NMR (100 MHz, CDCl₃, 298 K): δ=26.2, 26.3, 26.5, 26.5, 29.2, 29.4, 29.5, 29.6, 29.7, 29.7, 29.8, 29.9, 29.9, 65.4, 69.0, 69.1, 69.5, (7 signals missing, possibly because of signal overlap) 111.1, 111.8, 113.0, 114.2, 119.6, 126.6, 129.9, 133.8, 148.9, 149.6, 154.9, 191.0 (1 signals missing, possibly because of signal overlap); HR-MS (ESI): calcd for C₃₆H₅₄O₆⁺ [M]⁺, m/z 582.3920. found, m/z 582.3901.

Trialdehyde S11:

Following the procedure described above for S10, the reaction of the macrocycle S10 (1.88 g, 3.23 mmol) and Sc(OTf)₃ (79.6 mg, 0.16 mmol) in CHCl₃ (18.8 mL) at 70° C. for 16 h afforded a light-yellow solid S11 (215.2 mg, 12%).

¹H NMR (400 MHz, CDCl₃, 298 K): δ=1.20-1.56 (m, 88H), 1.68-1.88 (m, 24H), 3.47 (d, J=13.8 3H), 3.84-4.10 (m, 24H), 4.68 (d, J=13.6 Hz, 3H), 6.79-6.82 (m, 6H), 6.89-6.94 (m, 3H), 7.35-7.41 (m, 6H), 9.79 (s, 3H); ¹³C NMR (100 MHz, CDCl₃, 298 K): δ=26.2, 26.3, 26.4, 26.5, 29.2, 29.2, 29.4, 29.5, 29.5, 29.6, 29.6, 29.7, 29.8, 29.9, 36.4, 69.0, 69.1, 69.5, 69.7, (4 signals are missing, possibly because of signal overlapping), 111.0, 111.8, 116.0, 116.3, 126.6, 129.9, 132.2, 132.3, 147.9, 148.0, 149.6, 154.8, 191.0; HR-MS (ESI): calcd for C₁₀₈H₁₅₆O₁₅⁺ [M]⁺, m/z 1693.1444. found, m/z 1693.1444.

CTV3:

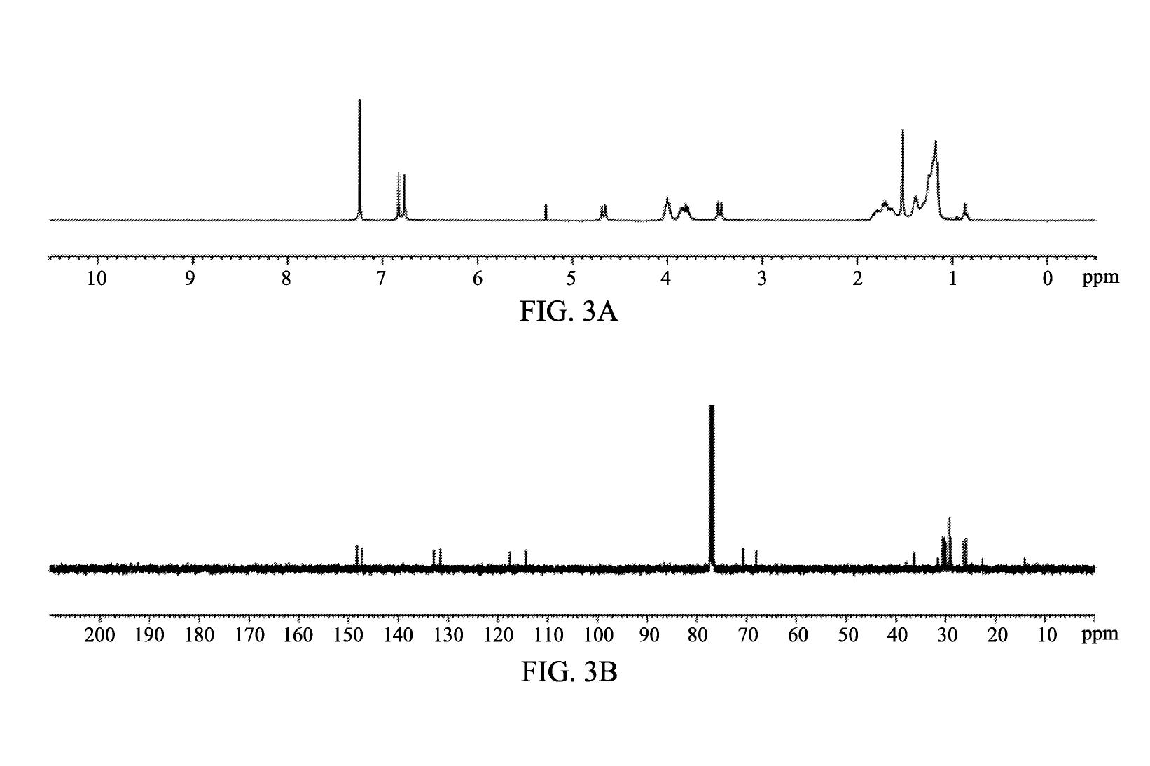

Following the procedure described above for S11, the reaction of the trialdehyde S11 (215 mg, 130 [symbol] mol) and NaBH₄ (19.2 mg, 0.51 mmol) in isopropyl alcohol (5.5 mL) and CH₂Cl₂ (5.5 mL) at room temperature for 16 h afford a white solid. The white solid was ten reacted with scandium triflate (81.4 mg, 165 [symbol] mol) in CHCl₃ (94 mL) at 60° C. for 3 days to afford a white solid CTV3 (30 mg, 14%). The ¹H NMR and ¹³C NMR spectra of CTV3 are shown in FIGS. 3A and 3B. All related spectral data are listed below.

¹H NMR (400 MHz, CDCl₃, 298 K): δ=1.12-1.44 (m, 84H), 1.57-1.87 (m, 24H), 3.45 (d, J=13.6 Hz, 6H), 3.77-3.90 (m, 12H), 3.96-4.06 (m, 12H), 4.67 (d, J=13.6, 6H), 6.77 (s, 6H), 6.83 (s, 6H); ¹³C NMR (100 MHz, CDCl₃, 298 K): δ=25.9, 26.3, 29.0, 29.2, 29.9, 30.2, 30.5, 31.6, 36.4, 68.0, 70.7, (1 signals are missing, possibly because of signal overlapping), 114.3, 117.6, 131.6, 132.9, 147.2, 148.3; HR-MS (ESI): calcd for C₁₀₈H₁₅₆O₁₂ [M]⁺, m/z 1645.1597. found, m/z 1645.1632.

### Synthesis of CTV4

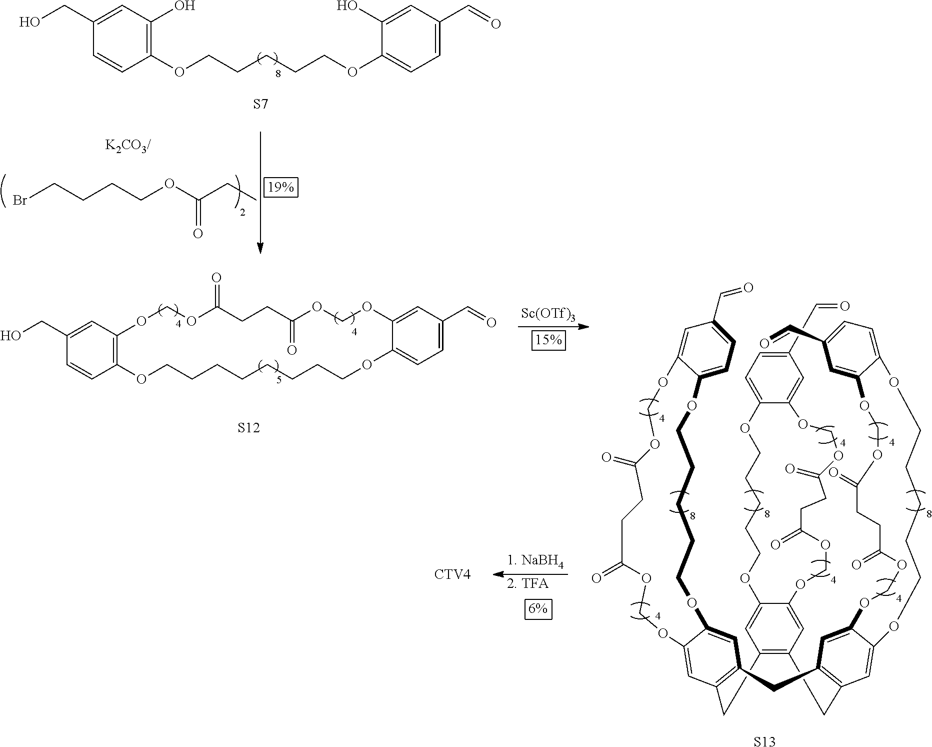

Macrocycle S12:

Following the procedure described above for S7, the reaction of the diol S7 (2.00 g, 4.50 mmol), bis(4-bromobutyl)succinate (1.75 g, 4.50 mmol) and K₂CO₃ (3.73 g, 26.99 mmol) in DMF (400 mL) at 60° C. for 6 days afforded a white solid S12 (0.59 g, 19%).

¹H NMR (400 MHz, CDCl₃, 298 K): δ=1.21-1.41 (m, 12H), 1.42-1.55 (m, 4H), 1.56-2.00 (m, 12H), 2.60 (s, 4H), 3.40-4.06 (m, 8H), 4.18-4.21 (m, 4H), 4.58 (s, 2H), 6.81-6.93 (m, 4H), 7.35 (d, J=1.6 Hz, 1H), 7.40 (dd, J=8.4, 1.8 Hz, 1H), 9.79 (s, 1H); ¹³C NMR (100 MHz, CDCl₃, 298 K): δ=26.2, 26.3, 29.2, 29.2, 29.4, 29.5, 29.5, 29.6, 29.6, 64.6, 64.7, 65.3, 68.7, 68.7, 69.0, 69.2, 110.9, 111.5, 113.1, 113.5, 119.8, 126.8, 129.6, 133.5, 148.7, 148.8, 149.0, 154.6, 171.9, 172.0, 190.7 (seven aliphatic signals are missing, possibly because of signal overlapping); HR-MS (ESI): calcd for C₃₈H₅₄O₁₀Na⁺ [M+Na]⁺, m/z 693.3615. found, m/z 693.3625.

Trialdehyde S13:

Following the procedure described above for S12, the reaction of the mono-alcohol S12 (1.82 g, 2.71 mmol) and Sc(OTf)₃ (67 mg, 0.140 mmol) in CHCl₃ (14 mL) at 70° C. for 16 h afforded the trialdehyde as a light-yellow oil S13 (274 mg, 15%).

¹H NMR (400 MHz, CDCl₃, 298 K): δ=1.16-1.52 (m, 42H), 1.67-1.94 (m, 42H), 2.56 (s, 12H), 3.46 (d, J=13.6 Hz, 3H), 3.78-4.00 (m, 12H), 4.00-4.09 (m, 12H), 4.09-4.24 (m, 12H), 4.67 (d, J=13.6, 3H), 6.79 (s, 3H), 6.80 (s, 3H), 6.91 (d, J=8.0 Hz, 3H), 7.35 (d, J=1.6 Hz, 3H), 7.39 (dd, J=8.2, 1.8 Hz, 3H), 9.79 (s, 3H); ¹³C NMR (100 MHz, CDCl₃, 298 K): δ=25.6, 25.7, 26.0, 26.1, 29.0, 29.2, 29.3, 29.3, 29.4, 29.4, 29.5, 36.3, 64.5, 68.5, 68.9, 69.2, 69.3, 110.8, 111.5, 115.6, 116.4, 126.9, 130.0, 132.0, 132.5, 147.5, 148.0, 149.1, 154.7, 172.1, 190.8 (six aliphatic and one aromatic signals are missing, possibly because of signal overlapping); HR-MS (ESI): calcd for C₁₁₄H₁₅₆O₂₇Na⁺[M+Na], m/z 1980.0732. found, m/z 1980.0764.

CTV4:

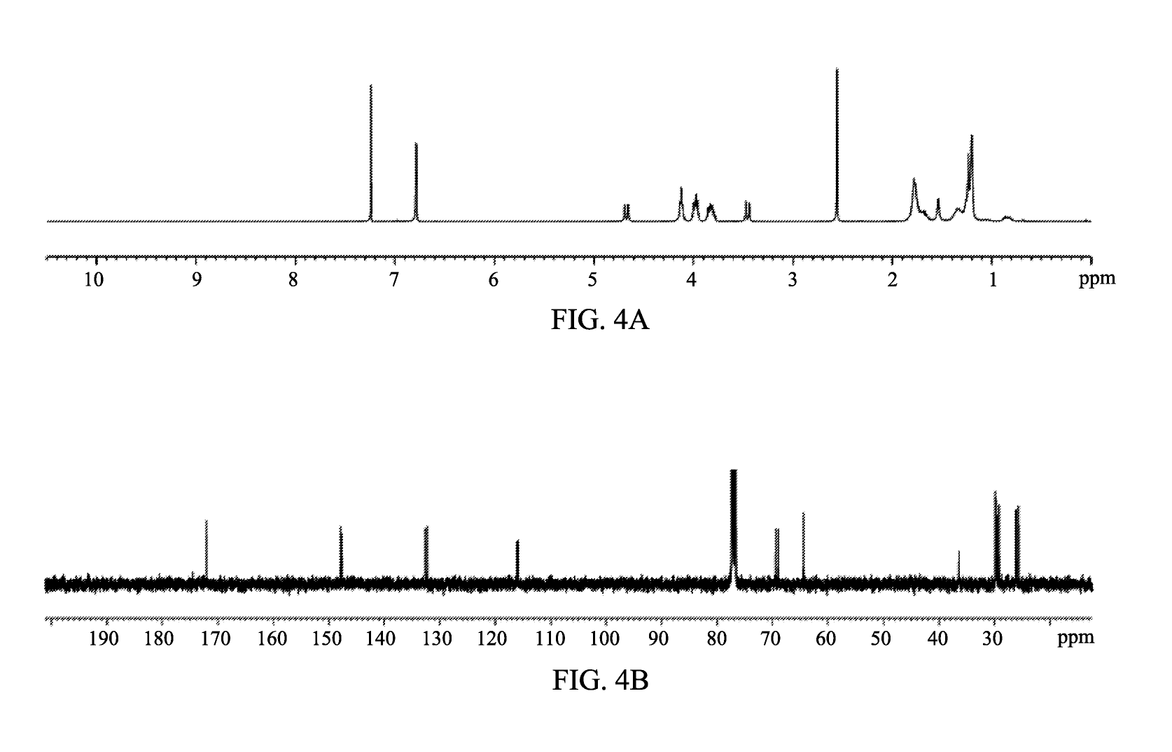

Following the procedure described above for S13, the reaction of the trialdehyde S13 (274 mg, 0.14 mmol) and NaBH₄ (16 mg, 0.42 mmol) in methanol (4.7 mL) and CH₂Cl₂ (9.3 mL) at −15° C. for 3.5 h afford a white solid. The white solid was then reacted with 10% TFA (15 mL) in CHCl₃ (102 mL) at room temperature for 2 days to afford a white solid CTV4 (16 mg, 6%). The ¹H NMR and ¹³C NMR spectra of CTV4 are shown in FIGS. 4A and 4B. All related spectral data are listed below.

¹H NMR (400 MHz, CDCl₃, 298 K): δ=1.13-1.45 (m, 42H), 1.59-1.89 (m, 42H), 2.56 (s, 12H), 3.45 (d, J=13.6 Hz, 6H), 3.75-3.91 (m, 12H), 3.91-4.05 (m, 12H), 4.06-4.21 (br, 12H), 4.67 (d, J=14.0, 6H), 6.78 (s, 6H), 6.79 (s, 6H); ¹³C NMR (100 MHz, CDCl₃, 298 K): δ=25.7, 26.1, 26.1, 29.2, 29.5, 29.7, 29.7, 29.8, 36.4, 64.4, 69.0, 69.4, 115.9, 116.1, 132.2, 132.5, 147.7, 147.9, 172.0; HR-MS (ESI): calcd for C₁₁₄H₁₅₆NaO₂₄⁺ [M+Na]⁺, m/z 1932.0884. found, m/z 1931.9346.

### Synthesis of CTV5

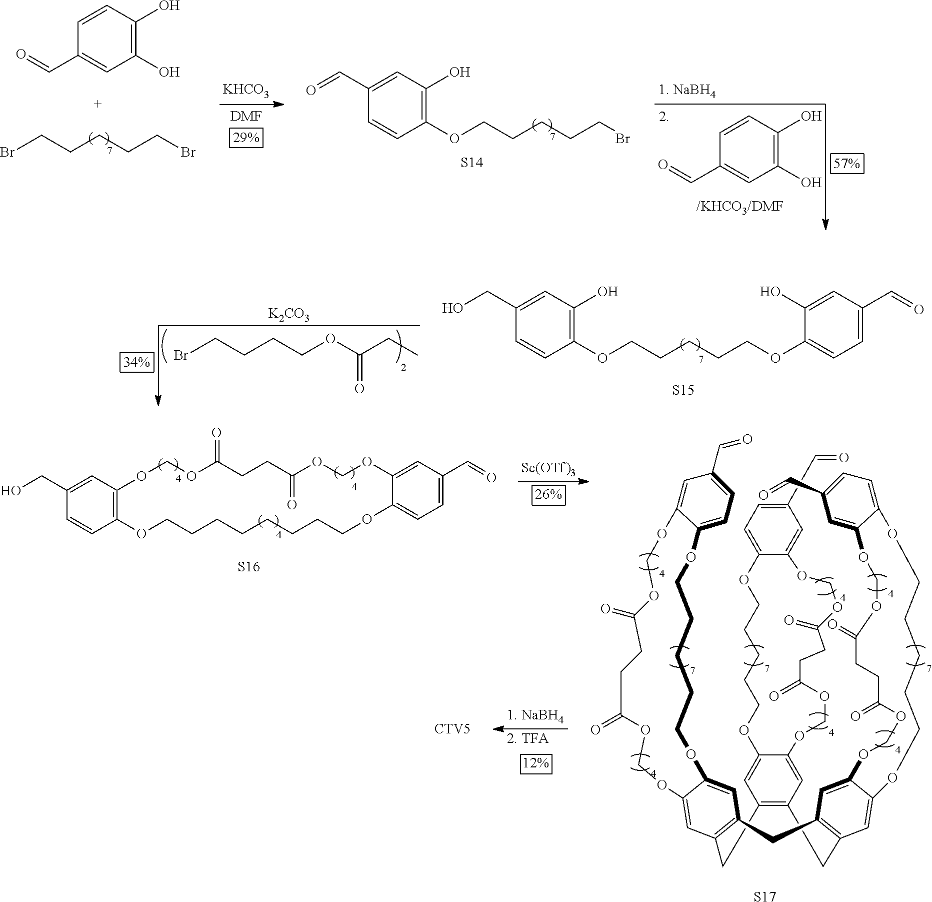

Aldehyde S14:

The reaction of potassium bicarbonate (7.16 g, 70.8 mmol), 3,4-dihydroxybenzaldehyde (8.15 g, 59.0 mmol), and 1,11-dibromoundecane (22.3 g, 70.8 mmol) in DMF (393 mL) at 60° C. for 2 days afforded aldehyde S14 as a white solid (6.38 g, 29%).

Mp: 60-61° C.; ¹H NMR (400 MHz, CDCl₃, 298 K): δ=1.28-1.48 (m, 14H), 1.79-1.87 (m, 4H), 3.38 (t, J=6.8 Hz, 2H), 4.11 (t, J=6.4 Hz, 2H), 5.75 (s, 1H), 6.93 (d, J=8.4 Hz, 1H), 7.39 (dd, J=8.4, 2 Hz, 1H), 7.42 (d, J=2 Hz, 1H), 9.81 (s, 1H); ¹³C NMR (100 MHz, CDCl₃, 298 K): δ=25.9, 28.1, 28.7, 29.0, 29.2, 29.3, 29.4, 29.4, 32.8, 34.0, 69.3, 110.9, 114.0, 124.4, 130.5, 146.2, 151.2, 190.9; HR-MS (ESI): calcd for C₁₈H₂₆O₃Br⁻ [M−H]⁻, m/z 369.1065. found, m/z 369.1062.

Alcohol S15:

Following the procedure described above for S14, the reaction of the aldehyde S14 (6.38 g, 17.19 mmol) and NaBH₄ (650 mg, 17.19 mmol) in methanol (30 mL) and CH₂Cl₂ (60 mL) at room temperature for 2 h afforded a white solid (1.89 g, 98%). The white solid was then reacted with 3,4-dihydroxybenzaldehyde (2.62 g, 18.94 mmol) and potassium bicarbonate (1.92 g, 18.94 mmol) in DMF (115 mL) at 60° C. for 2 days to afford a white solid S15 (4.19 g, 57%).

Mp: 94-97° C.; ¹H NMR (400 MHz, CDCl₃, 298 K): δ=1.29-1.48 (m, 14H), 1.75-1.87 (m, 4H), 4.01 (t, J=6.4 Hz, 2H), 4.11 (t, J=6.8 Hz, 2H), 4.56 (s, 2H), 5.68 (s, 1H), 5.81 (s, 1H), 6.78-6.82 (m, 2H), 6.91-6.93 (m, 2H), 7.38 (dd, J=8, 2 Hz, 1H), 7.41 (d, J=2 Hz, 1H), 9.81 (s, 1H); ¹³C NMR (100 MHz, CDCl₃, 298 K): δ=25.9, 25.9, 29.0, 29.2, 29.2, 29.3, 29.4, 29.4, 29.4, 65.1, 69.0, 69.3, 110.9, 111.6, 113.5, 114.1, 118.8, 124.4, 130.5, 134.2, 145.5, 145.9, 146.2, 151.3, 191.0; HR-MS (ESI): calcd for C₂₅H₃₃O₆⁻ [M−H]⁻, m/z 429.2277. found, m/z 429.2272.

Macrocycle S16:

Following the procedure described above for S15, the reaction of the diol S15 (3.64 g, 8.46 mmol), Bis(4-bromobutyl)succinate (3.28 g, 8.46 mmol) and K₂CO₃ (7.02 g, 50.77 mmol) in DMF (846 mL) at 60° C. for 5 days afforded a white solid S16 (1.83 g, 34%).

Mp: 125-127° C.; ¹H NMR (400 MHz, CDCl₃, 298 K): δ=1.29-1.33 (m, 10H), 1.42-1.49 (m, 4H), 1.70-1.89 (m, 12H), 2.59 (s, 4H), 3.93-4.05 (m, 8H), 4.17-4.20 (m, 4H), 4.56 (s, 2H), 6.80-6.93 (m, 4H), 7.35 (d, J=1.6 Hz, 1H), 7.40 (dd, J=8.4, 1.6 Hz, 1H), 9.79 (s, 1H); ¹³C NMR (100 MHz, CDCl₃, 298 K): δ=25.6, 25.7, 25.8, 25.9, 26.2, 26.2, 29.1, 29.2, 29.4, 29.4, 29.5, 29.6, 29.6, 64.5, 64.6, 65.2, 68.6, 68.7, 69.0, 69.2, 111.0, 111.6, 113.2, 113.6, 119.9, 126.9, 129.8, 133.7, 148.8, 149.0, 149.2, 154.8, 172.1, 172.1, 190.9; HR-MS (ESI): calcd for C₃₇H₅₂O₁₀Na⁺ [M+Na]⁺, m/z 679.3458. found, m/z 679.3466.

Trialdehyde S17:

Following the procedure described above for S16, the reaction of the macrocycle S16 (700 mg, 1.07 mmol) and Sc(OTf)₃ (26 mg, 0.053 mmol) in CHCl₃ (14 mL) at room temperature for 16 h afforded a light-yellow oil (180 mg, 26%).

¹H NMR (400 MHz, CDCl₃, 298 K): δ=1.32-1.48 (m, 36H), 1.69-1.86 (m, 42H), 2.56 (s, 12H), 3.45 (d, J=14 Hz, 3H), 3.83-4.05 (m, 24H), 4.14-4.19 (m, 12H), 4.67 (d, J=13.6 Hz, 3H), 6.79 (s, 3H), 6.79 (s, 3H), 6.91 (d, J=8.4 Hz, 3H), 7.35 (d, J=1.6 Hz, 3H), 7.39 (dd, J=8.4, 1.6 Hz, 3H), 9.79 (s, 3H); ¹³C NMR (100 MHz, CDCl₃, 298 K): δ=25.6, 25.6, 25.7, 26.0, 26.1, 26.2, 29.0, 29.4, 29.4, 29.4, 29.5, 29.6, 36.3, 64.4, 68.6, 69.0, 69.2, 69.4, 111.0, 111.6, 115.8, 116.5, 126.8, 129.8, 132.1, 132.6, 147.6, 148.0, 149.1, 154.7, 172.0, 190.7 (four aliphatic and one aromatic signals are missing, possibly because of signal overlapping); HR-MS (ESI): calcd for C₁₁₁H₁₅₀O₂₇Na⁺ [M+Na]⁺, m/z 1938.0261. found, m/z 1938.0191.

CTV5:

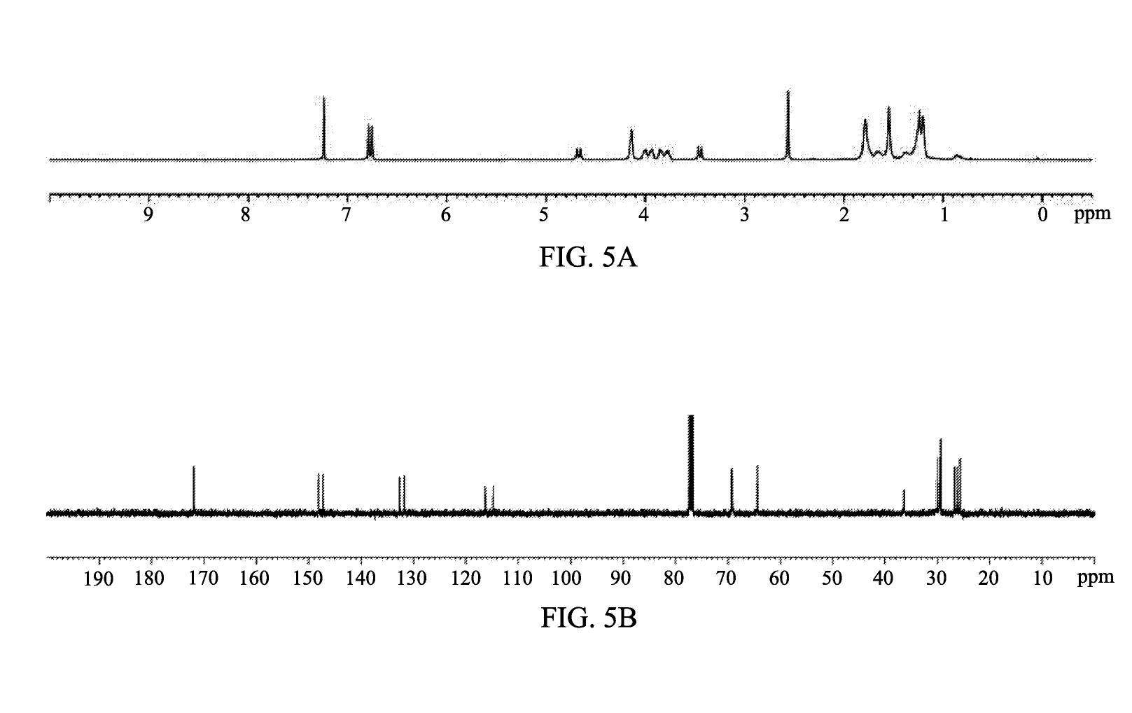

Following the procedure described above for S17, the reaction of the trialdehyde S17 (100 mg, 0.052 mmol) and NaBH₄ (4 mg, 0.1 mmol) in methanol (1.7 mL) and CH₂Cl₂ (3.4 mL) at −15° C. for 1.5 h afford a white solid. The white solid was then reacted with TFA (8 mL) in CHCl₃ (57 mL) at room temperature for 2 days to afford a white solid CTV5 (12 mg, 12%). The ¹H NMR and ¹³C NMR spectra of CTV5 are shown in FIGS. 5A and 5B. All related spectral data are listed below.

¹H NMR (400 MHz, CDCl₃, 298 K): δ=1.20-1.38 (m, 36H), 1.55-1.78 (m, 42H), 2.56 (s, 12H), 3.45 (d, J=13.6 Hz, 6H), 3.75-3.85 (m, 12H), 3.93-4.03 (m, 12H), 4.14 (br, 12H), 4.67 (d, J=13.6, 6H), 6.75 (s, 6H), 6.79 (s, 6H); ¹³C NMR (100 MHz, CDCl₃, 298 K): δ=25.7, 26.1, 26.7, 29.3, 29.4, 29.6, 30.0 (one signal missing, possibly because of signal overlap), 36.3, 64.4, 69.1, 69.3, 114.8, 116.3, 131.8, 132.6, 147.3, 148.1, 172.0; HR-MS (ESI): calcd for C₁₁₁H₁₅₀NaO₂₄⁺ [M+Na]⁺, m/z 1890.0414. found, m/z 1890.0342.

### Synthesis of CTV6

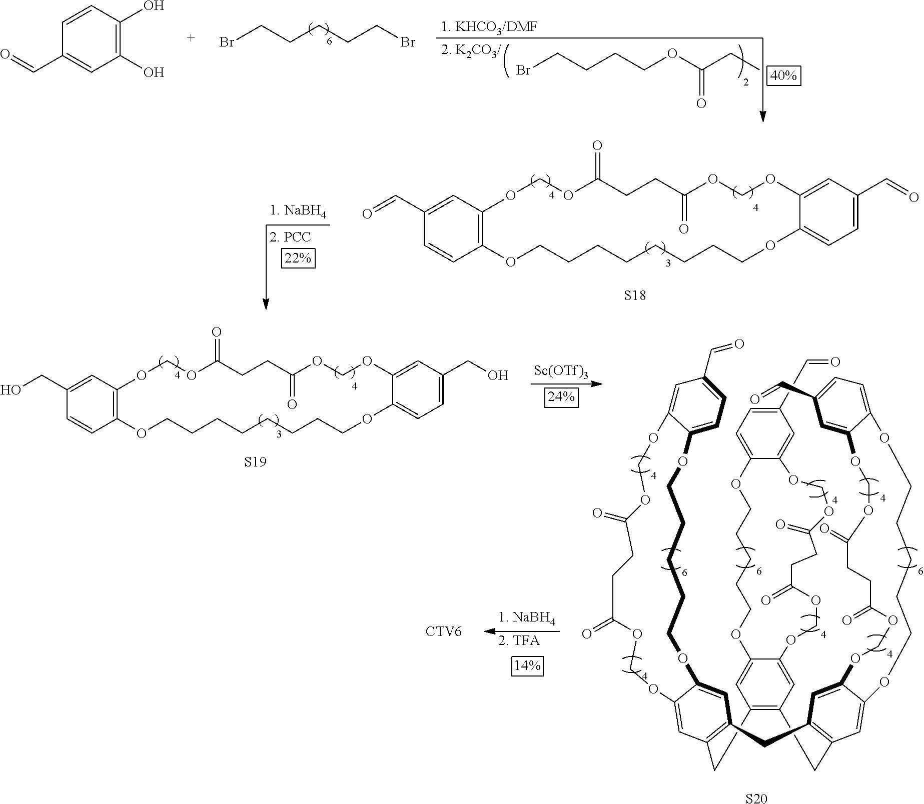

Macrocycle S18:

The reaction of the potassium bicarbonate (1.22 g, 12.03 mmol), 3,4-dihydroxybenzaldehyde (1.66 g, 12.03 mmol), and 1,10-dibromodecane (1.64 g, 5.47 mmol) in DMF (11 mL) at 65° C. for 2 days afforded a white solid. The white solid was then reacted with (bis(4-bromobutyl) succinate) (1.66 g, 4.28 mmol) and K₂CO₃ in DMF (427 mL) to afford a white solid S18 (3.64 g, 40%).

¹H NMR (400 MHz, CDCl₃, 298 K): δ=1.33-1.50 (m, 12H), 1.79-1.87 (m, 12H), 2.59 (s, 4H), 4.02-4.05 (m, 8H), 4.18-4.21 (m, 4H), 6.92 (d, J=8.4 Hz, 2H), 7.35 (d, J=1.6 Hz, 2H), 7.40 (dd, J=8, 1.2 Hz, 2H), 9.80 (s, 2H); ¹³C NMR (100 MHz, CDCl₃, 298 K): δ=25.6, 25.9, 26.2, 29.1, 29.1, 29.4, 29.5, 64.6, 68.7, 69.0, 110.9, 111.6, 126.9, 129.8, 149.2, 154.7, 172.1, 190.9; HR-MS (ESI): calcd for C₃₆H₄₈O₁₀Na⁺ [M+Na]⁺, m/z 663.3145. found, m/z 663.3177.

Alcohol S19:

Following the procedure described above for S18, the reaction of the dialdehyde S18 (2.13 g, 3.33 mmol) and NaBH₄ (126 mg, 3.33 mmol) in methanol (50 mL) and CH₂Cl₂ (50 mL) at 0° C. for 2 h afforded a white solid. The following reaction of the white solid, pyridinium chlorochromate (669 mg, 3.10 mmol) and 4-Å molecular sieves (2 g) in CH₂Cl₂ (124 mL) at room temperature for 3 h afforded a white solid S19 (712 mg, 22%).

¹H NMR (400 MHz, CDCl₃, 298 K): δ=1.32 (br, 8H), 1.43-1.48 (m, 4H), 1.74-1.89 (m, 12H), 2.59 (s, 4H), 3.93-4.07 (m, 8H), 4.19-4.21 (m, 4H), 4.57 (s, 2H), 6.80-6.93 (m, 4H), 7.35 (d, J=1.6 Hz, 1H), 7.40 (dd, J=8, 1.6 Hz, 1H), 9.80 (s, 1H); ¹³C NMR (100 MHz, CDCl₃, 298 K): δ=25.6, 25.8, 25.9, 25.9, 26.1, 26.2, 29.0, 29.2, 29.3, 29.4, 29.5, 29.5, (two signals missing, possibly because of signal overlap), 64.6, 64.6, 65.2, 68.7, 68.7, 69.0, 69.2, 110.9, 111.6, 113.1, 113.5, 119.9, 126.9, 129.8, 133.7, 148.8, 149.0, 149.2, 154.8, 172.1, (one signal missing, possibly because of signal overlap), 190.9; HR-MS (ESI): calcd for C₃₆H₅₀O₁₀Na⁺ [M+Na]⁺, m/z 665.3302. found, m/z 665.3344.

Trialdehyde S20:

Following the procedure described above for S19, the reaction of the mono-alcohol S19 (500 mg, 0.78 mmol) and Sc(OTf)₃ (19 mg, 0.039 mmol) in CHCl₃ (8 mL) at 70° C. for 16 h afforded the trialdehyde as a light-yellow oil S20 (118 mg, 24%).

¹H NMR (400 MHz, CDCl₃, 298 K): δ=1.30-1.47 (m, 30H), 1.69-1.85 (m, 42H), 2.55 (s, 12H), 3.45 (d, J=13.6 Hz, 3H), 3.82-4.04 (m, 24H), 4.14-4.20 (m, 12H), 4.67 (d, J=13.6 Hz, 3H), 6.78 (s, 3H), 6.79 (s, 3H), 6.91 (d, J=8.4 Hz, 3H), 7.34 (s, 3H), 7.39 (d, J=8.4, 3H), 9.79 (s, 3H); ¹³C NMR (100 MHz, CDCl₃, 298 K): δ=25.5, 25.7, 25.8, 25.9, 26.1, 26.2, 29.0, 29.0, 29.3, 29.3, 29.4, 29.4, 29.5, 36.3, 64.5, 64.5, 68.6, 68.9, 69.2, 69.3, 110.8, 111.5, 115.6, 116.4, 126.8, 129.7, 132.0, 132.5, 147.5, 148.0, 149.1, 154.7, 172.0, 172.0, 190.8 (one aliphatic signal is missing, possibly because of signal overlap); HR-MS (ESI): calcd for C₁₀₈H₁₄₄O₂₇Na⁺ [M+Na]⁺, m/z 1895.9793. found, m/z 1895.9826.

CTV6:

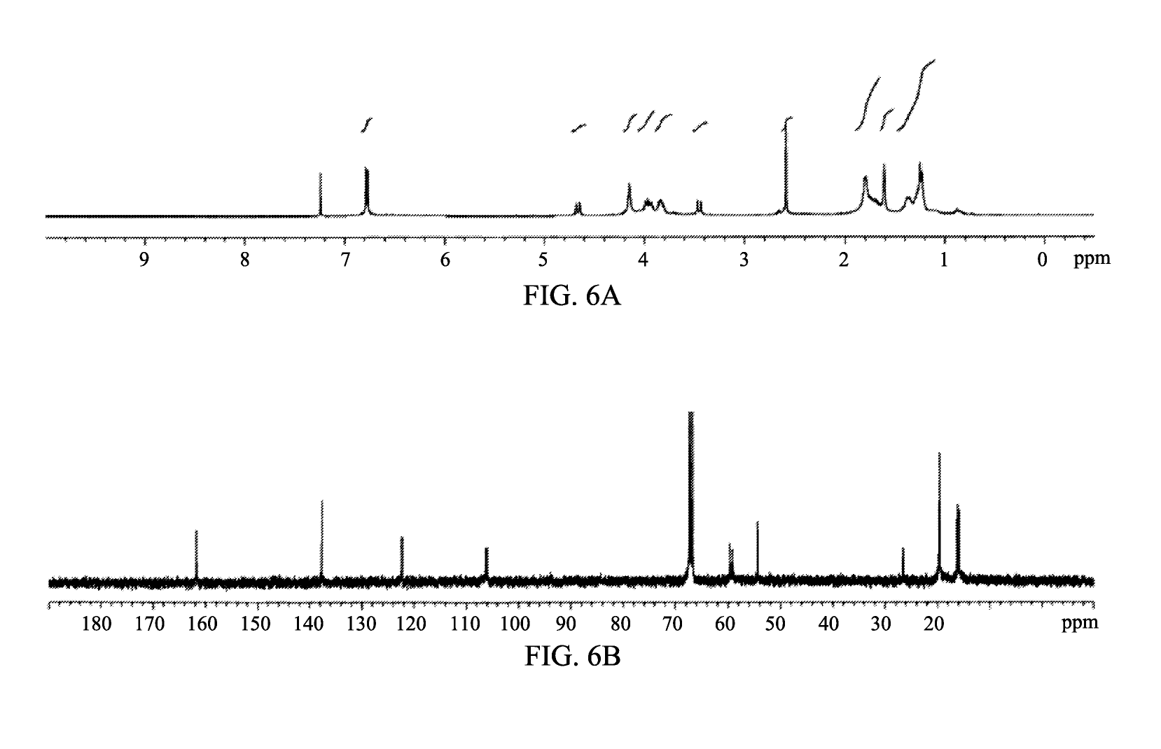

Following the procedure described above for S20, the reaction of the trialdehyde S20 (58 mg, 0.031 mmol) and NaBH₄ (4 mg, 0.047 mmol) in methanol (1.5 mL) and CH₂Cl₂ (1.5 mL) at 0° C. for 1.5 h afford a white solid. The white solid was then reacted with TFA (3 mL) in CHCl₃ (50 mL) at room temperature for 2 days to afford a white solid CTV6 (8 mg, 14%). The ¹H NMR and ¹³C NMR spectra of CTV6 are shown in FIGS. 6A and 6B, All related spectral data are listed below.

¹H NMR (400 MHz, CDCl₃, 298 K): δ=1.23-1.39 (m, 30H), 1.64-1.82 (m, 42H), 2.58 (s, 12H), 3.45 (d, J=13.6 Hz, 6H), 3.79-3.87 (m, 12H), 3.91-4.00 (m, 12H), 4.15-4.16 (br, 12H), 4.66 (d, J=13.2 Hz, 6H), 6.77 (s, 6H), 6.78 (s, 6H); ¹³C NMR (100 MHz, CDCl₃, 298 K): δ=25.8, 26.1, 26.3, 29.5, 29.6, 29.7, 29.8, 36.5, 64.4, 69.1, 69.6, 116.0, 116.3, 132.2, 132.4, 147.6, (one signal missing, possibly because of signal overlap), 171.7; HR-MS (ESI): calcd for C₁₀₈H₁₄₄NaO₂₄⁺ [M+Na]⁺, m/z 1847.9945. found, m/z 1848.0018.

### Synthesis of Fullerene CTV Complexes

Fullerene CTV complexes, comprising fullerene⊙CTV hemicarceplexes, formed by various cyclotriveratrylene-based molecular cages, and various fullerene guests or derivatives thereof are disclosed below. Generally, fullerene⊙CTV complexes are formed by mixing a fullerene or a fullerene mixture with a CTV host in a less-polar solvent being capable of dissolving fullerene CTV complexes at or above room temperature, such as 25-80° C.

The meaning of “a complex” above is a supramolecular host-guest assembly, in which the fullerene guest is located inside the cavity of the CTV host. The meaning of “a hemicarceplex” above is a room temperature-isolatable complex. The meaning of “room temperature” is without heating at all. The meaning of “a fullerene” comprises an unmodified fullerene and a derivative thereof. The meaning of “a fullerene's derivative” comprises a fullerene molecule trapping an atom, an ion, a molecule or a metal cluster therein, such as Sc₃N@C₈₀.

Generally, the size of the inner space of the CTV host is strongly related to its shortest linking spacer(s). The size of the CTV host's openings can be adjusted by changing the length of its longer linking spacer(s). Thus, the selectivity of fullerene for entering the CTV host and its entering easiness can be controlled by adjusting the lengths of both linking spacers.

### Formation of C₆₀ CTV1 Complex

C₆₀ CTV1 complex was synthesized by mixing equimolar of the CTV1 and purified C₆₀ in CDCl₂CDCl₂.

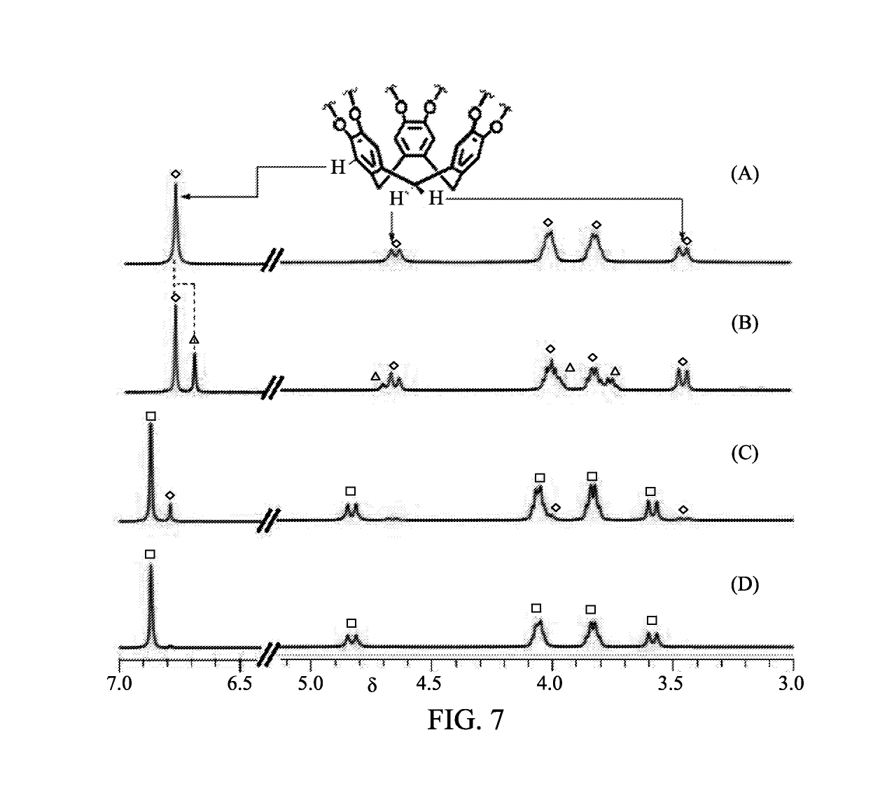

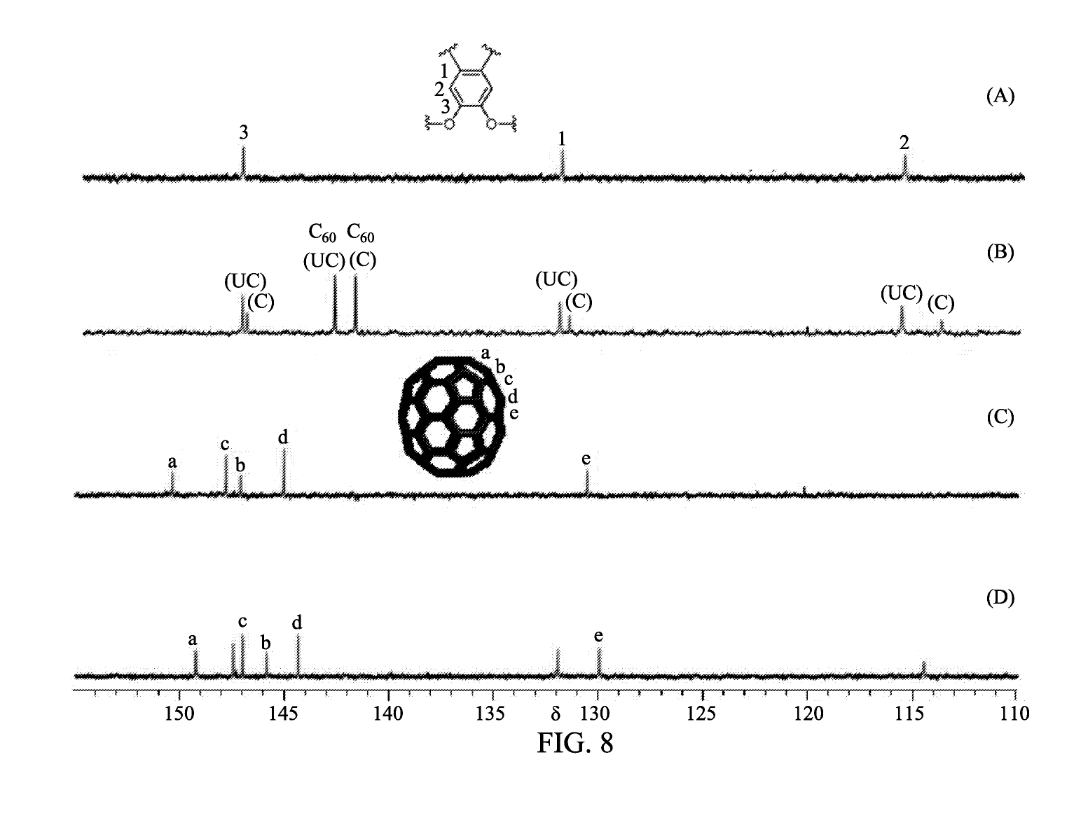

The ¹H NMR and ¹³C NMR spectra of the equimolar mixture of C₆₀ and CTV1 are respectively shown in FIGS. 7B and 8B. In FIG. 7B, the descriptors (c) and (uc) respectively refer to the complexed and uncomplexed states of the corresponding components. For comparison, the ¹H NMR and ¹³C NMR spectra of CTV1 are also respectively shown in FIGS. 7A and 8A.

In FIG. 7B, the ¹H NMR spectrum of the 3 mM equimolar mixture of C₆₀ and CTV1 shows a new set of signals corresponding to the C₆₀ CTV1 complex.

Therefore, C₆₀ CTV1 complex was not sufficiently stable for isolation through column chromatography at ambient temperature, and thus cannot be called as hemicarceplex. However, the ¹H NMR spectrum of the equimolar mixture of C₆₀ and CTV1 suggests that the rates for C₆₀ guest entry into and exit from the internal cavity of CTV1 were slow on the timescale of ¹H NMR spectroscopy at 400 MHz.

In FIG. 8B, the ¹³C NMR spectra of the 2.5 mM equimolar mixture of C₆₀ and CTV1 shows an upfield shifting of the signals of C₆₀ within C₆₀ CTV1 complex. This implied that the spherical fullerene could also be positioned favorably within the cavity of CTV1.

### Synthesis of C₇₀ CTV1 Hemicarceplex

C₇₀ CTV1 hemicarceplex was synthesized by the following steps. First, equimolar of the CTV1 and C₇₀ were mixed in CDCl₂CDCl₂. Then, the mixture was heated at 60° C. for 48 hours to form C₇₀ CTV1 hemicarceplex. The ¹H NMR spectrum of the 3 mM equimolar mixture of C₇₀ and CTV1 is shown in FIG. 7C.

Another solution of the CTV1 (40 mg, 23 μmol) and C₇₀ (19.4 mg, 23 μmol) in CHCl₂CHCl₂ (5 mL) was stirred at 60° C. for 24 hours and then the solvent was evaporated under reduced pressure. The residue was purified chromatographically (SiO₂; CS₂ then CH₂Cl₂/hexanes, 1:1 in volume ratio) to afford a black solid of C₇₀ CTV1 hemicarceplex (32 mg, 54%).

All related spectral data of the purified C₇₀ CTV1 hemicarceplex are provided below. Mp: >300° C.; ¹H NMR (400 MHz, CDCl₂CDCl₂, 298 K): δ=1.12-1.57 (m, 96H), 1.68-1.88 (m, 24H), 3.58 (d, J=13 Hz, 6H), 3.78-3.88 (m, 12H), 4.01-4.11 (m, 12H), 4.84 (d, J=13 Hz, 6H), 6.87 (s, 12H); ¹³C NMR (100 MHz, C₂D₂Cl₄, 298 K): δ=26.8, 29.7, 29.9, 30.1, 30.2, 36.9, 68.7, 114.5, 130.0, 131.9, 144.3, 145.8, 147.0, 147.4, 149.2; HR-MS (ESI): calcd for C₁₈₄H₁₆₈O₁₂⁺ [M]⁺, m/z 2569.2536. found, m/z 2569.2704.

Accordingly, unlike complex C₆₀ CTV1, the C₇₀ CTV1 could be purified chromatographically, and thus can be called as a hemicarceplex. An electrospray ionization (ESI) mass spectrum of the purified C₇₀ CTV1 revealed intense peaks at m/z 2569.3 corresponding to the ions [C₇₀ CTV1]⁺. The good matches between the observed and calculated isotope patterns for the ion support the successful synthesis of the hemicarceplex C₇₀ CTV1.

The ¹H NMR and ¹³C NMR spectra of the purified C₇₀ CTV1 hemicarceplex is shown in FIGS. 7D and 8D. In FIG. 8D, the ¹³C NMR spectrum of the isolated hemicarceplex C₇₀ CTV1 displays all five signals belonging to C₇₀, shifted upfield by 0.6-1.2 ppm relative to those of the free C₇₀ (FIG. 8C), suggesting encapsulation of spheroidal C₇₀ within the cavity of CTV1.

### Synthesis of C₇₀⊙CTV2 Hemicarceplex

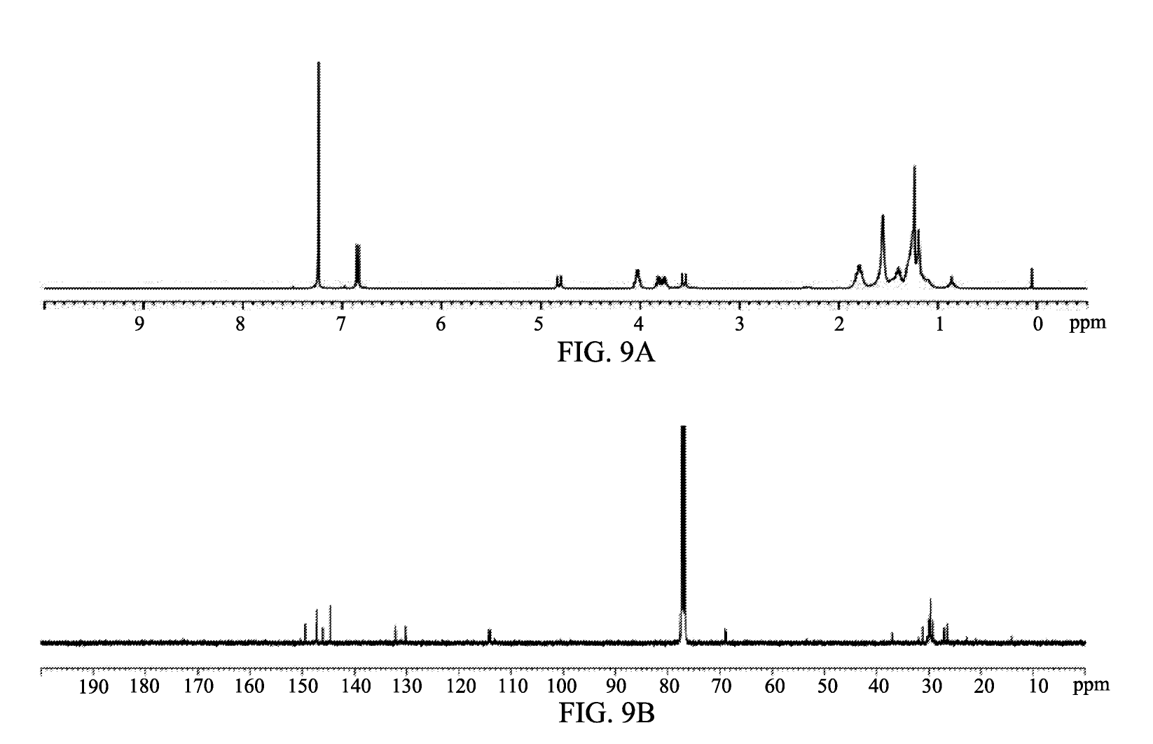

C₇₀⊙CTV2 hemicarceplex was synthesized by the following steps. First, CTV2 (40 mg, 23.6 μmol) and C₇₀ (40 mg, 48 μmol) were mixed in CHCl₂CHCl₂ (2 mL) and stirred at 60° C. for 2 days. Then, the residue was purified chromatographically (SiO₂; CS₂ then CH₂Cl₂/hexanes, 4:1) to afford C₇₀⊙CTV2 hemicarceplex as a black solid (28.6 mg, 46%). The ¹H NMR and ¹³C NMR spectra of the purified C₇₀⊙CTV2 hemicarceplex is shown in FIGS. 9A and 9B.

An electrospray ionization (ESI) mass spectrum of the purified C₇₀⊙CTV2 revealed intense peaks at m/z 2528.2144 corresponding to the ions [C₇₀⊙CTV2+H]⁺.

Comparing CTV1 and CTV2, since the carbon number of three alkyl chains between two cyclotriveratrylenes of CTV2 were decreased from 12 to 11, the size of the inner space and openings of CTV2 were both reduced, too.

However, complex C₆₀⊙CTV2 still not stable enough to be chromatographically isolated in pure, thus, cannot be considered as a hemicarceplex. C₇₀⊙CTV1 and the C₇₀⊙CTV2 can be purified chromatographically, and thus can be called as hemicarceplex.

All related spectral data of the purified C₇₀ CTV2 hemicarceplex are provided below. Mp: >300° C. (dec.); ¹H NMR (400 MHz, CDCl₃, 298 K): δ=1.12-1.50 (m, 90H), 1.72-1.88 (m, 26H), 3.56 (d, J=13.6 Hz, 6H), 3.71-3.85 (m, 12H), 3.99-4.07 (m, 12H), 4.84 (d, J=13.6 Hz, 6H), 6.83 (s, 6H), 6.86 (s, 6H); ¹³C NMR (100 MHz, CDCl₃, 298 K): δ=26.4, 27.0, 29.2, 29.7, 29.8, 29.9, 30.0, 30.1, 30.4, 31.1, 37.0, 68.8, 68.9, 113.9, 114.3, 130.2, 132.1, 132.2, 144.6, 146.1, 147.2, 147.3, 147.4, 149.4; HR-MS (ESI): calcd for C₁₈₄H₁₆₈O₁₂⁺ [M+H]⁺, m/z 2528.2066 found, m/z 2528.2144.

### Synthesis of C₆₀⊙CTV3 Hemicarceplex

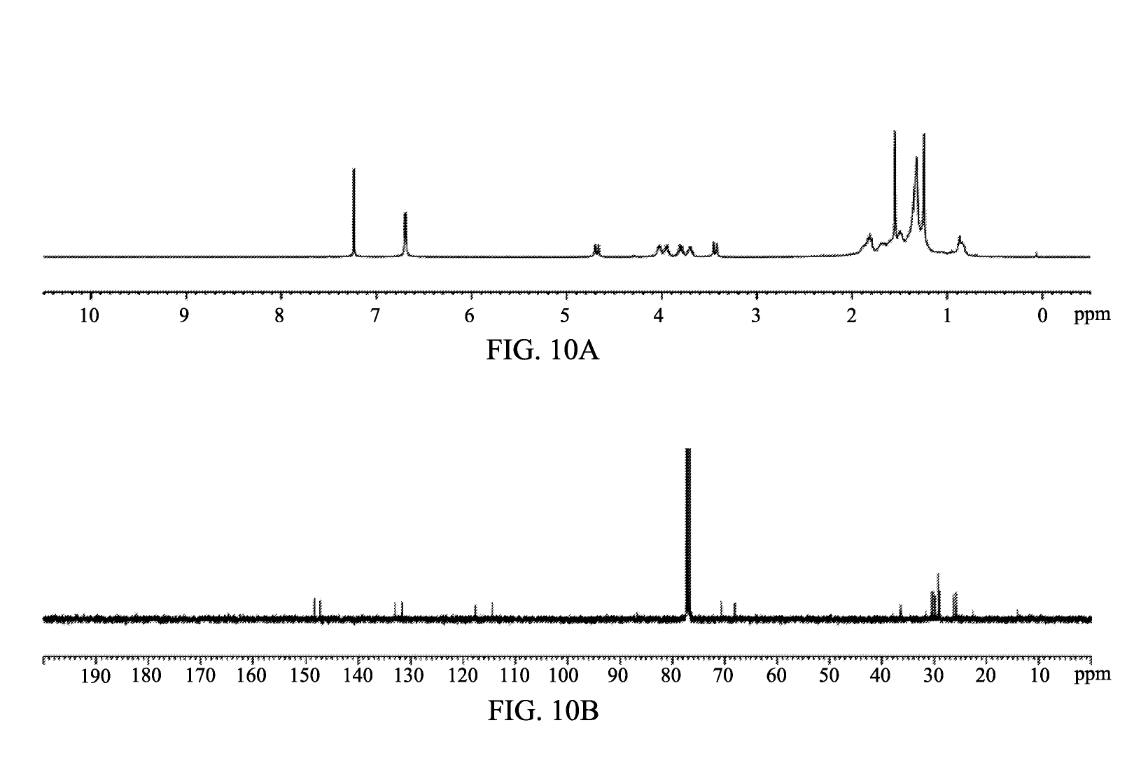

C₆₀⊙CTV3 hemicarceplex was synthesized by the following steps. First, CTV3 (10 mg, 6.07 μmol) and C₆₀ (10 mg, 13.9 μmol) were mixed in CHCl₂CHCl₂ (2 mL) and stirred at 50° C. for 20 h. Then, the residue was purified chromatographically (SiO₂; CS₂ then CH₂Cl₂/hexanes, 4:1) to afford C₆₀⊙CTV3 hemicarceplex as a black solid (4.3 mg, 30%). The ¹H NMR and ¹³C NMR spectra of the purified C₆₀⊙CTV3 hemicarceplex are shown in FIGS. 10A and 10B.

Comparing CTV2 and CTV3, since the carbon number of three alkyl chains between two cyclotriveratrylenes of CTV3 were further decreased from 11 to 10, the size of the inner space and openings of CTV3 was further reduced, too. Thus, it appeared that the more sizable C₇₀ is not capable to enter the cavity of CTV3 but the smaller C₆₀ can form stable hemicarceplex C₆₀⊙CTV3 with CTV3.

All related spectral data of the purified C₆₀ CTV3 hemicarceplex are provided below. ¹H NMR (400 MHz, CDCl₃, 298 K): δ=1.29-1.53 (m, 84H), 1.77-1.93 (m, 24H), 3.44 (d, J=13.6 Hz, 6H), 3.66-3.86 (m, 12H), 3.91-4.07 (m, 12H), 4.69 (d, J=14 Hz, 6H), 6.69 (s, 6H), 6.71 (s, 6H); ¹³C NMR (200 MHz, CDCl₃, 298 K): δ=25.8, 27.0, 28.3, 28.8, 29.1, 29.2, 30.1, 30.8, 31.0, 37.0, 68.7, 68.8, 114.1, 114.2, 131.9, 132.0, 142.1, 147.3, 147.4; HR-MS (ESI): calcd for C₁₆₈H₁₅₆O₁₂⁺ [M]⁺, m/z 2365.1597 found, m/z 2365.1649.

As C₇₀ CTV1 and C₇₀ CTV2, C₆₀ CTV3 could also be purified chromatographically, and thus can be called as hemicarceplex. An electrospray ionization (ESI) mass spectrum of the purified C₆₀ CTV3 revealed intense peaks at m/z 2365.1649 corresponding to the ions [C₆₀ CTV3]⁺.

### Synthesis of Sc₃N@C₈₀⊙CTV4 Hemicarceplex

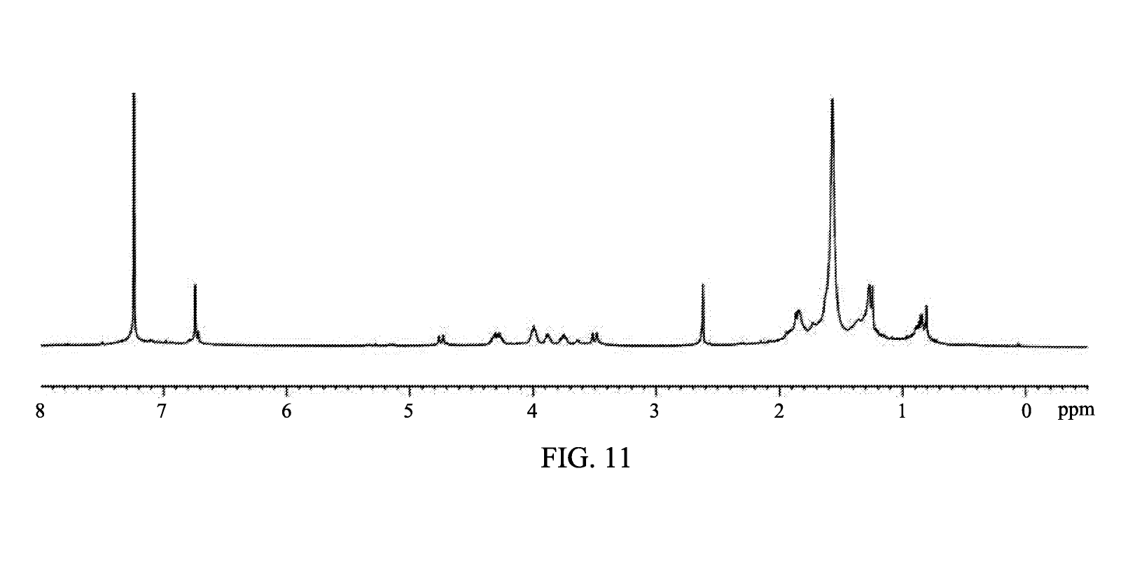

Sc₃N@C₈₀⊙CTV4 hemicarceplex was synthesized by the following steps. First, equimolar of the CTV4 and Sc₃N@C₈₀ were mixed in CDCl₂CDCl₂ to form an equimolar mixture (6 mM). Then, the mixture stirred at room temperature for 20 hours to form Sc₃N@C₈₀⊙CTV4 hemicarceplex. The ¹H NMR spectrum of the purified Sc₃N@C₈₀⊙CTV4 hemicarceplex is shown in FIG. 11. An electrospray ionization (ESI) mass spectrum of the purified Sc₃N@C₈₀⊙CTV4 revealed intense peaks at m/z 3020.1055 corresponding to the ions [Sc₃N@C₈₀⊙CTV4+H]⁺.

All related spectral data of the purified Sc₃N@C₈₀⊙CTV4 hemicarceplex are provided below. ¹H NMR (400 MHz, CDCl₃, 298 K): δ=1.11-1.45 (m, 42H), 1.77-2.07 (m, 42H), 2.62 (s, 12H), 3.50 (d, J=13.6 Hz, 6H), 3.71-3.80 (m, 6H) 3.84-3.91 (m, 6H), 3.96-4.04 (m, 12H), 4.23-4.36 (m, 12H), 4.74 (d, J=13.2, 6H), 6.74 (s, 12H); HR-MS (ESI): calcd for C₁₉₄H₁₅₇NO₂₄Sc₃ [M+H]⁺, m/z 3020.1745. found, m/z 3020.1055.

### The Formation of C₆₀⊙CTV5 and C₇₀⊙CTV5 Complexes

C₆₀⊙CTV5 and C₇₀⊙CTV5 complexes were synthesized by mixing equimolar of the CTV5 and purified C₆₀ and C₇₀ in CDCl₂CDCl₂, respectively.

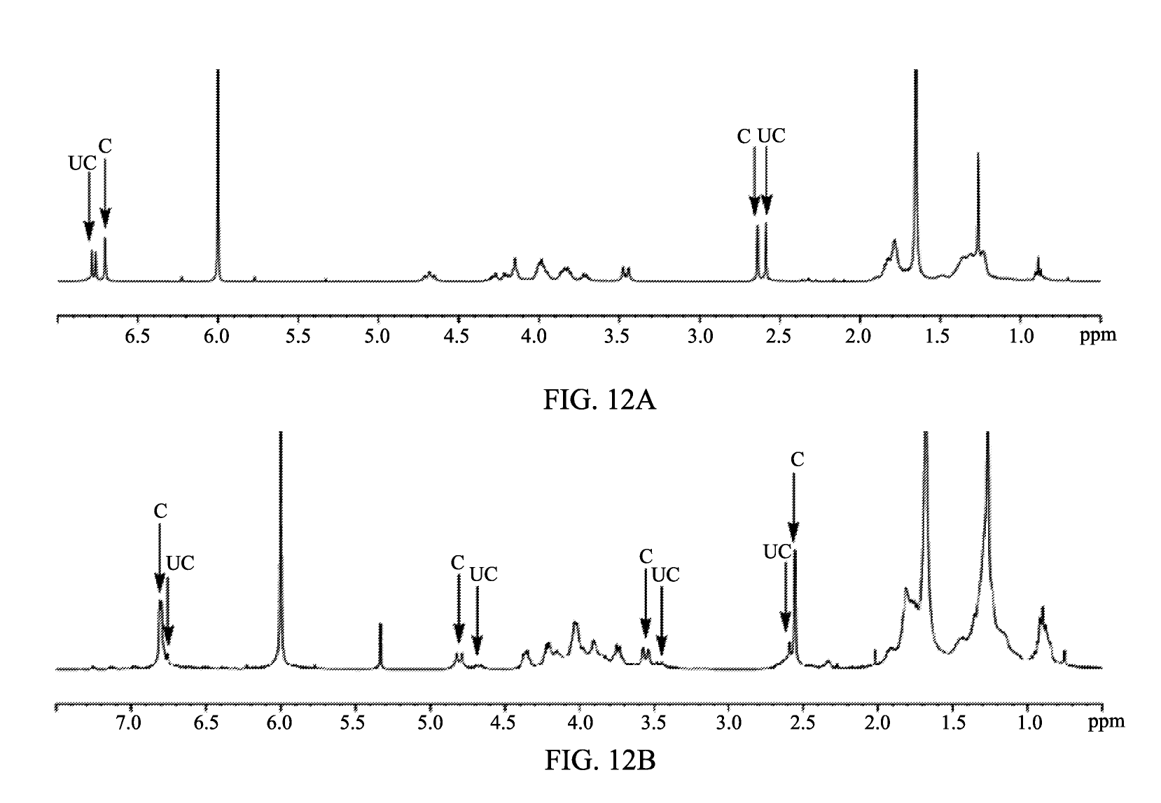

The ¹H NMR spectra of the equimolar mixture of CTV5 to C₆₀ and C₇₀ are respectively shown in FIGS. 12A and 12B. In FIG. 12, the descriptors (c) and (uc) respectively refer to the complexed and uncomplexed states of the corresponding components.

Comparing CTV2 and CTV5, the atom numbers of the three alkyl chains between two cyclotriveratrylenes were the same (11 atoms), but the atom numbers of the other three chains were increase from 12 to 14, the size of the openings of the CTV5 was increased. Therefore, C₆₀ and C₇₀ could both enter the inner space of the CTV5 but the complexes formed are not stable enough to be isolated through column chromatography and cannot be called as hemicarceplexes.

### The Formation of C₆₀⊙CTV6 Complexes

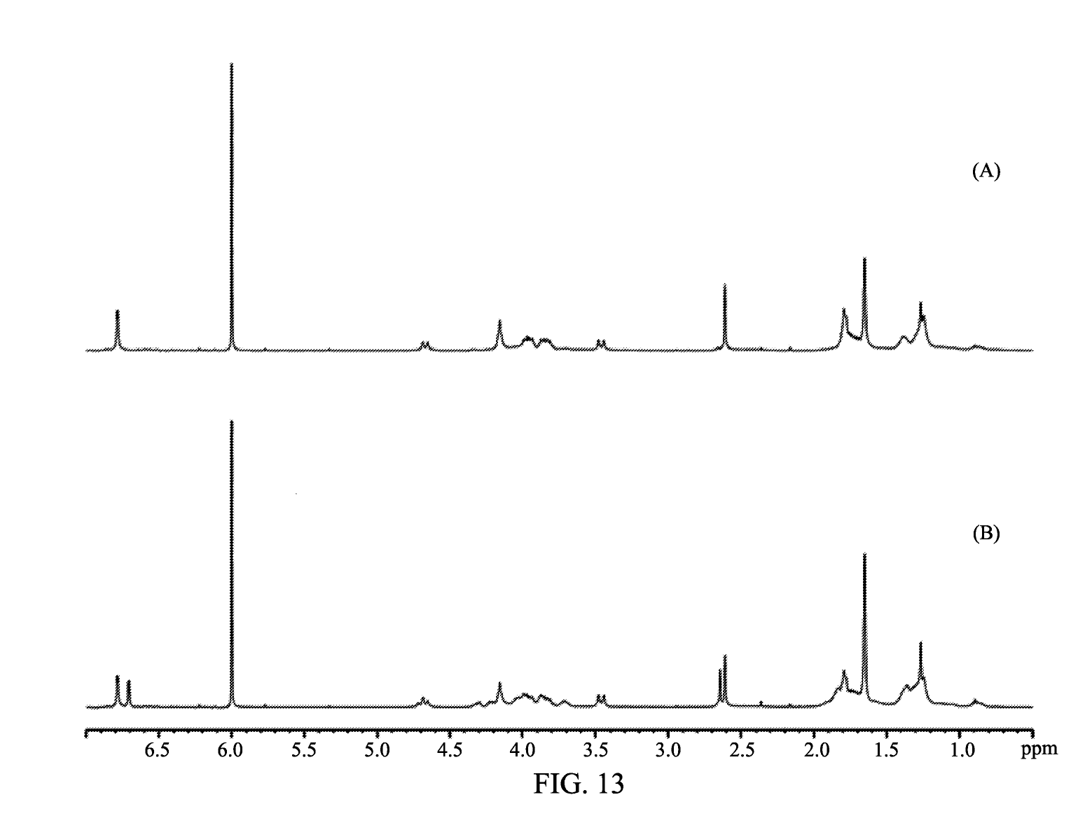

C₆₀⊙CTV6 complexes were synthesized by mixing equimolar of the CTV6 and C₆₀ in CDCl₂CDCl₂. The ¹H NMR spectra of the equimolar mixture of CTV6 and C₆₀ are shown in FIG. 13B. For comparison, the ¹H NMR spectrum of CTV6 is also shown in FIG. 13A.

In FIG. 13B, the ¹H NMR spectrum of the 4 mM equimolar mixture of C₆₀ and CTV6 shows a new set of signals corresponding to the C₆₀ CTV6 complex.

Comparing CTV6 and CTV5, the atom numbers of the three alkyl chains between two cyclotriveratrylenes were decreased from 11 to 10, but the atom numbers of the other three chains remains the same (14 atoms), the size of the molecular openings of the CTV6 was, thus, reduced. Therefore, C₆₀ but not C70 could enter the inner space of the CTV6, however, the complexes formed are not stable enough to be isolated through column chromatography.

### Kinetic Data of C₆₀ CTV1 and C₇₀ CTV1

### Complexation of the CTV1 with C₆₀

Experiments were performed in CDCl₂CDCl₂ using an equimolar (3 mM) mixture of the CTV1 and C₆₀ at 25° C.

In this experiment, a simplified second-order rate equation (1) shown below was used to calculate an association rate constant (kₐ) of forming C₆₀ CTV1 complex at the early stage of complexation.

kₐ=(1/[Aₜ]−1/[A₀])/t={1/([A₀]−[Pₜ])−1/[A₀]}/t (1)

The initial concentration of free CTV, [A₀], and the free C₆₀, [B₀], were both 3 mM. [Aₜ] is the concentration of the free CTV1 at time t, and [Pₜ] is the concentration of the C₆₀ CTV1 complex at time t. [Aₜ] and [Pₜ] were determined based on the integration values of the signals at δ 6.80 (Hₚ, s, 12H) and δ 6.70 (H_{p′}, s, 12H), respectively. [Aₜ] could also be determined based on [A₀]−[Pₜ]. Accordingly, based on a plot of 1/[Aₜ]−1/[A₀] against t (s) at 298 K, the association rate constant (kₐ) was obtained by calculating the slope of the plot. The obtained association rate constant (kₐ) was 1.68×10⁻¹ M⁻¹s⁻¹.

The half-life time of the complexation reaction (t_{1/2}) was given by equation (2) below. The calculated half-life time of the complexation reaction (t_{1/2}) was 33.1 min.

t_{1/2}=1/(kₐ[A₀]) (2)

The value of ΔG^{‡} (kcal mol⁻¹) was calculated using the relationship shown in equation (3) below, where R, h, and k_{B} are the gas, Planck, and Boltzmann constants, respectively. The calculated ΔG^{‡} was 18.49 kcal mol⁻¹.

ΔG^{‡}=−RT ln(kh/k_{B}T) (3)

The equilibrium constant (Kₐ) of forming C₆₀ CTV1 complex at 25° C. was also determined. ¹H NMR spectrum (400 MHz, CDCl₂CDCl₂, 298 K) of an equimolar (C_{M}=3 mM) mixture of C₆₀ and the CTV1 after heating at 298 K for 7 days was used to determine the equilibrium constant (Kₐ) of forming C₆₀ CTV1 complex. The integration values for the signals of the free (I_{f}, 6.8 ppm) and complexed (I_{c}, 6.7 ppm) species are 1.016 and 0.843, respectively. Therefore, the equilibrium constant (Kₐ) of forming C₆₀ CTV1 complex was determined by using equation (4) below to be 506 M⁻¹.

Kₐ=I_{c}(I_{f}+I_{c})/(I_{f}²C_{M}) (4)

### Dissociation of the Hemicarceplex C₇₀ CTV1

The dissociation experiments were performed using constant concentrations of the hemicarceplex C₇₀ CTV1 (3 mM) in CDCl₂CDCl₂, ds-toluene, CDCl₃, CDCl₃/CD₃CN (95:5 and 90:10 in volume ratio), and CDCl₃/CD₃NO₂ (95:5 and 90:10 in volume ratio). ¹H NMR spectra were recorded at regular intervals during the experiment. Because of the poor solubility of the CTV1 in toluene, trichloroethene was added as an internal standard to determine the concentration of the hemicarceplex during the dissociation.

The reverse reaction can be ignored during the early stages of the first-order decomplexation event. Therefore, using the first-order rate law shown in equation (5) below, the dissociation rate constants (k_{d}) were determined at the early stages of decomplexation from the slopes of the straight lines in the plots of ln([A₀]/[Aₜ]) against t (s) at 25° C. The concentration of the C₇₀ CTV1 hemicarceplex, [Pₜ], was determined from the integration of signals at ca. δ 6.90 (Hₚ, s, 12H).

k_{d}=ln([P₀]/[Pₜ])/t (5)

The half-life time of the decomplexation reaction (t_{1/2}) was given by equation (6) below.

t_{1/2}=ln(2/k_{d}) (6)

The values of ΔG^{‡} (kcal mol⁻¹) were also calculated using the relationship shown in equation (3).

The obtained dissociation rate constants (k_{d}), ΔG^{‡}, and t_{1/2} are listed in Table 1 below. From the dissociation rate constants (k_{d}) listed in Table 1, it can be known that the dissociation rate constant was decreased as the polarity of the solvent system was increased from CDCl₂CDCl₂ to CDCl₃/CD₃CN (90:10). This is because of the lipophilicity of C₇₀, which make the dissociation state of the hemicarceplex C₇₀ CTV1 to be more unstable.

TABLE 1
The obtained dissociation rate constants (k_{d}), ΔG^{‡}, and t_{1/2} for
the hemicarceplex C₇₀ CTV1 dissociated in various solvent system.

| | | | ΔG^{‡} |
|---|---|---|---|
| Solvent | k_{d} (s⁻¹) | t_{1/2} (h) | (kcal mol⁻¹) |
| d₈-toluene | 6.5 ± 0.7 × 10⁻⁵ | 3.0 ± 0.3 | 23.2 ± 0.1 |
| CDCl₂CDCl₂ | 6.3 ± 0.7 × 10⁻⁶ | 30.6 ± 3.3 | 24.6 ± 0.1 |
| CDCl₃ | 5.1 ± 0.5 × 10⁻⁶ | 37.8 ± 3.3 | 24.7 ± 0.1 |
| CDCl₃/CD₃NO₂ (95:5) | 5.0 ± 0.5 × 10⁻⁶ | 38.5 ± 3.8 | 247.8 ± 0.1 |
| CDCl₃/CD₃CN (95:5) | 4.3 ± 0.4 × 10⁻⁶ | 44.8 ± 4.0 | 24.7 ± 0.1 |
| CDCl₃/CD₃NO₂ (90:10) | 3.6 ± 0.3 × 10⁻⁶ | 53.5 ± 4.0 | 25.0 ± 0.1 |
| CDCl₃/CD₃CN (90:10) | 3.2 ± 0.3 × 10⁻⁶ | 60.2 ± 4.9 | 24.9 ± 0.1 |

Similarly, the relatively rapid dissociation of the hemicarceplex C₇₀ CTV1 in toluene-d₈ was because of C₇₀ stabilized more in the dissociated state than in the complex. Moreover, because of the poor solubility of the CTV1 in toluene-d₈, a white solid precipitated from the red solution during dissociation of the hemicarceplex C₇₀ CTV1 in this solvent. Correspondingly, the ¹H NMR spectra revealed a gradual decrease in the intensity of signals belonging to the hemicarceplex, but without the appearance of any signals for the free CTV1. Accordingly, the relatively rapid dissociation rate and the precipitation of the free CTV1 from the red solution of the hemicarceplex C₇₀ CTV1 in toluene-d₈ suggested that toluene would be a good choice of solvent for dissociating the hemicarceplex into its free components on a practical scale.

The equilibrium constant (Kₐ) of forming C₇₀ CTV1 complex at 25° C. was also determined. ¹H NMR spectrum (400 MHz, CDCl₂CDCl₂, 298 K) of the mixture obtained from the decomplexation of C₇₀⊙CTV1 (C_{M}=3 mM) at 298 K after 10 days was used to determine equilibrium constant (Kₐ) of forming C₇₀ CTV1 complex. The integration values for the signals of the free (I_{f}, 6.8 ppm) and complexed (I_{c}, 6.9 ppm) species are 0.324 and 1.000, respectively. Therefore, the equilibrium constant (Kₐ) of forming C₇₀ CTV1 complex was determined by using equation (4) above to be 4204 M⁻¹. In addition, the association rate constant (kₐ) was determined to be 0.026 M⁻¹·s⁻¹. The determination methods were similar to the methods mentioned for the C₆₀ CTV1 complex above, and hence omitted here.

### Isolating Fullerenes from Fullerene Extracts by Using Fullerene CTV Hemicarceplexes

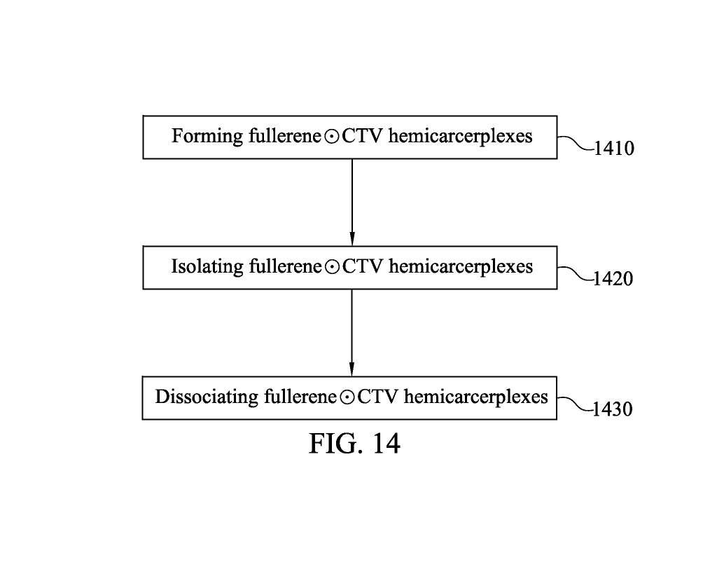

Fullerenes or derivatives thereof can be isolated from their mixtures in high purity through the following steps: (1) selectively generating a fullerene⊙CTV hemicarceplex in solution, (2) isolating the fullerene⊙CTV hemicarceplexes by column chromatography, and (3) dissociating the fullerene⊙CTV hemicarceplexes to release the pure fullerenes. FIG. 14 is a process flow diagram of isolating fullerenes without using crystallization or HPLC.

In the step 1410 of FIG. 14, fullerene CTV hemicarceplexes has to be formed first. In step 1420, the fullerene CTV hemicarceplexes are then isolated by using column chromatography. In step 1430, the isolated fullerenes are obtained by dissociating the fullerene CTV hemicarceplexes. The related details of each step are described below.

In step 1410 of FIG. 14, fullerene CTV hemicarceplexes are formed by mixing a CTV host and a fullerene mixture, such as a fullerene extract, in a first solvent at a first temperature to form a first mixture solution. Then, the first mixture solution is concentrated under a reduced pressure to obtain a first solid residue.

The first solvent can dissolve both fullerenes and CTV host and do not have strong tendency to enter and compete with fullerenes in occupying the inner space of the CTV host. For example, the first solvent can majorly contain CS₂, CH₂Cl₂, CHCl₃ or CHCl₂CHCl₂ but is not limited thereto.

The formation of fullerene CTV hemicarceplexes can be inspected by either ¹H or ¹³C NMR, such as those NMR spectra shown in FIGS. 7-11 discussed above. Accordingly, the lowest first temperature has to be sufficiently high to see that new NMR signals corresponding to the fullerene CTV hemicarceplexes appear within few hours. For example, the first temperature can be room temperature to 60° C., such as 40° C., but is not limited thereto.

Moreover, the needed reaction time can also be determined by either 1H or ¹³C NMR. When the new NMR signals corresponding to the fullerene CTV hemicarceplexes grow to reach a maximum strength, the reaction can be stopped.

Next, a second solvent is added to the first solid residue to form a suspended solution. Then, the suspended solution is filtered to obtain filtrate of the suspended solution.

The CTV host and the fullerene CTV hemicarceplexes have better solubility in the second solvent than the free fullerenes. Therefore, most of the free fullerenes can be filtered off, and the filtrate contains the fullerene CTV hemicarceplexes. For example, the second solvent can majorly contain CH₂Cl₂, CHCl₃ or CHCl₂CHCl₂, but is not limited thereto.

In step 1420 of FIG. 14, the filtrate of the suspended solution in step 1410 is concentrated and then loaded onto a column of silica gel to prepare for a subsequent column chromatography. Then, a third solvent, a fourth solvent, and a fifth solvent were sequentially used to elute the free fullerenes, fullerene⊙CTV hemicarceplexes, and free CTV hosts from the column. Since the polarity of the free fullerenes, fullerene⊙CTV hemicarceplexes, and free CTV hosts are generally in an increasing order, the polarity of the third solvent, the fourth solvent, and the fifth solvents for eluting the molecules above are better also generally in an increasing order.

Accordingly, since the third solvent is used to remove any uncomplexed and/or dissociated fullerenes from the column, the third solvent has to be capable of dissolving free fullerenes. For examples, the third solvent can majorly contain CS₂, benzene, toluene or dichlorobenzene but is not limited thereto.

The fourth solvent is used to isolate the fullerene CTV hemicarceplexes from the column. Therefore, the fourth solvent has to be capable of dissolving the fullerene CTV hemicarceplexes. For example, the fourth solvent can majorly contains CH₂Cl₂ or CHCl₃, such as CH₂Cl₂ and hexane mixed in a volume ratio of 3:2, but is not limited thereto.

The fifth solvent is used to elute the free CTV host from the column, so that the free CTV host can be recover for the next use. Therefore, the fifth solvent has to be capable of dissolving the free CTV host. For example, the fifth solvent can majorly contains CH₂Cl₂ or CHCl₃, such as CH₂Cl₂ and MeOH mixed in a volume ratio of 49:1, but is not limited thereto.

In step 1430 of FIG. 14, the portion of the eluate containing the fullerene⊙CTV hemicarceplexes is concentrated, and a sixth solvent is then added to dissociate the fullerene CTV hemicarceplexes at a second temperature. In the best scenario, free fullerenes have good solubility in the sixth solvent, and the CTV host has a poor solubility in the sixth solvent. Therefore, the released fullerenes and fullerene CTV hemicarceplexes are still dissolved in the sixth solvent, but the free CTV host is precipitated as solid. Otherwise, the dissociated free fullerenes can still be separated from free cage and the hemicarceplexes by chromatographic methods. Accordingly, the sixth solvent can majorly contain CS₂, CH₂Cl₂, CHCl₃, CHCl₂CHCl₂, benzene, toluene, or dichlorobenzene, for example, but is not limited thereto.

Since the dissociation reaction is the reverse reaction the hemicarceplexe formation reaction, the dissociation reaction may also need heating. The lowest temperature for the second temperature also can be determined by NMR spectra when the NMR signals corresponding to the fullerene CTV hemicarceplexes decrease within few hours. For examples, the second temperature can be higher than room temperature, such as 30° C. to 80° C., but is not limited thereto.

Next, the solution of the sixth solvent was concentrated, and a seventh solvent is then added. For separating the released fullerenes and fullerene CTV hemicarceplexes, the released fullerenes have a poor solubility in the seventh solvent, and the fullerene CTV hemicarceplexes have a good solubility in the seventh solvent. Therefore, the seventh solvent can majorly contain CH₂Cl₂, or CHCl₃, for example, but is not limited thereto.

Since the seventh solvent has poor solubility for the free fullerenes, the free fullerenes are precipitated. Hence, a simple filtering step can obtain the free fullerenes.

### Experiment 1

### Using CTV1 to Isolate C₇₀ from Fullerene Extract; Small Scale

(1) Forming C₇₀⊙CTV1 Hemicarceplex:

The CTV1 (50 mg) and the fullerene extract above (300 mg, purchased from SES Research) were dissolved in CHCl₂CHCl₂ (5 mL) and stirred at various temperatures for various periods of time for Examples 1-6. The organic solvent was removed under reduced pressure and the residue was dissolved in CH₂Cl₂ (40 mL). After filtration, the solvent was evaporated under reduced pressure to obtain various amounts of solid for Examples 1-6. The related data above were listed in Table 2 below.

TABLE 2
Related data of forming C₇₀⊙CTV1 hemicarceplex

| Examples | Stirring temp. (° C.) | Stirring time (h) | Solid amount (mg) |
|---|---|---|---|
| 1 | 40 | 16 | 86.4 |
| 2 | 40 | 16 | 101.1 |
| 3 | 40 | 16 | 111.2 |
| 4 | 50 | 16 | 72.8 |
| 5 | 40 | 40 | 91.4 |
| 6 | 40 | 16 | 98.4 |

(2) Using Column Chromatography to Isolate C₇₀⊙CTV1 Hemicarceplex:

The obtained solid above was purified through column chromatography (5 g of SiO₂). The eluents of CS₂, CH₂Cl₂/hexane (3:2 in volume ratio) and CH₂Cl₂/MeOH (49:1 in volume ratio) were sequentially used to sequentially elute free fullerenes, C₇₀⊙CTV1, and free CTV1 from the column. The eluate portions of the CH₂Cl₂/hexanes (3:2 in volume ratio) of Examples 1-6 were then respectively evaporated to obtain various amounts of C₇₀⊙CTV1. The using amounts of each eluent and the obtained amounts of C₇₀⊙CTV1 are all listed in Table 3 below.

TABLE 3
Related data of column chromatography

| | Eluents (mL) | | | |
|---|---|---|---|---|
| | | CH₂Cl₂/ | | C₇₀⊙CTV1 |
| Examples | CS₂ | hexane (3:2) | CH₂Cl₂/MeOH (49:1) | (mg) |
| 1 | 50 | 250 | 100 | 37.2 |
| 2 | 50 | 250 | 50 | 33.1 |
| 3 | 50 | 400 | 50 | 32.2 |
| 4 | 50 | 200 | 50 | 30.0 |
| 5 | 50 | 250 | 50 | 38.4 |
| 6 | 50 | 250 | 50 | 37.3 |

(3) Obtaining C₇₀ by Dissociating C₇₀⊙CTV1

The obtained C₇₀⊙CTV1 hemicarceplex was then dissolved in toluene (4 mL) and heated at a temperature for 12 h to dissociate the C₇₀⊙CTV1 hemicarceplex. The toluene solution was centrifuged to yield upper toluene solution and white precipitate. The upper toluene solution containing the free C₇₀ was removed via pipette. Another charge of toluene (5 mL) was added to wash the white solid of CTV1 to wash down the residue free C₇₀ from the white solid. Then, the two toluene solutions were mixed and centrifuged again to take the upper toluene solution. The white solid was recycled as the free CTV1.

The residue obtained after concentrating the combined toluene solutions was suspended in CH₂Cl₂ (5 mL) again to dissolve the highly soluble C₇₀⊙CTV1 hemicarceplex. However, the free C₇₀ formed red precipitate in the CH₂Cl₂. Therefore, the C₇₀ red precipitate in the CH₂Cl₂ was obtained by centrifuging the CH₂Cl₂ suspension and then drying, and the black CH₂Cl₂ solution was then dried to recycle C₇₀⊙CTV1 hemicarceplex.

The composition of the purchased fullerene extract and the purity of C₇₀ was determined through HPLC analysis (Cosmosil-packed 5PBB analytical column, 4.6×250 mm; mobile phase, toluene; UV detection, 285 nm; elution rate, 1 mL min⁻¹). Accordingly, the compositions of the purchased fullerene extract and the purity of C₇₀ were determined by dividing the integration value of the corresponding signal by the total integration values of the C₆₀, C₇₀, C₇₆, C₇₈, and C₈₄ signals.

Accordingly, the dissociation temperatures and the obtained amounts and purity of C₇₀ for Examples 1-6 were listed in Table 4, and the amounts of the recycled C₇₀⊙CTV1 hemicarceplex and recovered CTV1 for Examples 1-6 were listed in Table 5. The compositions of the purchased fullerene extract and the purified C₇₀ of Example 1 are listed in Table 6 below.

TABLE 4
Related data of dissociating C₇₀⊙CTV1 hemicarceplex

| | Dissociation | | Purity of |
|---|---|---|---|
| Examples | temp. (° C.) | Obtained C₇₀ (mg) | obtained C₇₀ (%) |
| 1 | 30 | 7.1 | 99.1 |
| 2 | 30 | 6.5 | 99.0 |
| 3 | 30 | 6.7 | 99.1 |
| 4 | 30 | 8.2 | 93.5 |
| 5 | 30 | 8.0 | 96.4 |
| 6 | 40 | 8.3 | 96.7 |

TABLE 5
Recovered amounts of the C₇₀⊙CTV1 hemicarceplex and CTV1

| Examples | C₇₀⊙CTV1 (mg) | CTV1 (mg) |
|---|---|---|
| 1 | 15.0 | 30.3 |
| 2 | 12.8 | 30.5 |
| 3 | 12.3 | 30.6 |
| 4 | — | 27.6 |
| 5 | — | 30.4 |
| 6 | — | 31.6 |

TABLE 6
Analyzing compositions of the purchased fullerene extract and
the purified C₇₀ of Examples 1-6 by HPLC

| Examples | C₆₀ (%) | C₇₀ (%) | C₇₆ (%) | C₇₈ (%) | C₈₄ (%) |
|---|---|---|---|---|---|
| 1 | 0.03 | 99.1 | 0.83 | 0 | 0 |
| 2 | 0.14 | 99.0 | 0.88 | 0 | 0 |
| 3 | 0.03 | 99.1 | 0.90 | 0 | 0 |
| 4 | 0.21 | 93.5 | 2.75 | 0.13 | 0 |
| 5 | 0.35 | 96.4 | 2.99 | 0.27 | 0 |
| 6 | 0.07 | 96.7 | 1.66 | 0.13 | 0 |
| Fullerene extract | 65.97 | 24.02 | 1.75 | 1.85 | 2.62 |

### Experiment 2

### Using CTV1 to Isolate C₇₀ from Fullerene Extract; Large Scale

After obtaining consistent results when repeating the isolation as shown above, the scale was increased to ten-fold of the previous small scale experiments.

The CTV1 (500 mg) and the fullerene extract (3.0 g) were dissolved in CHC₂CHCl₂ (50 mL) and stirred at 40° C. for 16 h. The organic solvent was removed under reduced pressure and the residue was dissolved in CH₂Cl₂ (250 mL). After filtration, the solvent was evaporated under reduced pressure to afford a solid (950 mg), which was purified through column chromatography [SiO₂ (25 g); eluent: CS₂ (250 mL) followed by CH₂Cl₂/hexane, 3:2 (1550 mL), and CH₂Cl₂/MeOH, 49:1 (600 mL)].

The hemicarceplex (366.1 mg) obtained was then dissolved in toluene (20 mL) and heated at 30° C. for 12 h. The toluene solution was removed via pipette after centrifugation. Another charge of toluene (20 mL×2) was added to wash the white solid and then the mixture was centrifuged again. The white solid was recycled as the free CTV1. The residue obtained after concentrating the combined toluene phases was suspended in CH₂Cl₂ (20 mL), in which the hemicarceplex C70⊙CTV1 is highly soluble, to form a red precipitate of C₇₀, which was collected through centrifugation. The solid C₇₀ was then resuspended in CH₂Cl₂ (20 mL×2), centrifuged, separated from the solvent, and dried (72.6 mg). The purity of C₇₀, determined through HPLC analysis, was 99.0%. The black CH₂Cl₂ solution was then concentrated to recycle 96.4 mg of the hemicarceplex C70⊙CTV1. The total amount of recovered CTV1 was 360 mg (72% recovery).

The recycled hemicarceplex (96.4 mg) was redissolved in toluene (10 mL) and heated at 30° C. for 12 h. The suspension was centrifuged to afford the CTV1 as a white precipitate. The organic solution was removed via pipette and concentrated to give a black solid, which was dissolved in CH₂Cl₂ (10 mL) and centrifuged. The solid C₇₀ was then resuspended in CH₂Cl₂ (10 mL×2), centrifuged, separated from the solvent, and dried (6.6 mg). The purity of the C₇₀, determined through HPLC analysis, was 92.6%. The black CH₂Cl₂ solution was concentrated to obtain 78.6 mg of the recycled hemicarceplex C70⊙CTV1. The amount of recycled CTV1 was 13.8 mg (26% recovery).

Accordingly, the compositions of the large-scale purified C70 obtained in the first round and second round of isolation process and the purchased fullerene extract are listed in Table 7 below. The percentages of each component were determined by the HPLC method mentioned in the small scale experiment above.

TABLE 7
Analyzing the compositions of the large-scale purified C70
obtained in the first round and second round of isolation process by HPLC

| | composition | | | | |
|---|---|---|---|---|---|
| | C₆₀ (%) | C₇₀ (%) | C₇₆ (%) | C₇₈ (%) | C₈₄ (%) |
| First round | 0.03 | 99.0 | 0.90 | 0.10 | 0 |
| Second round | 0.05 | 92.6 | 6.00 | 0.65 | 0 |
| Fullerene extract | 65.97 | 24.02 | 1.75 | 1.85 | 2.62 |

Accordingly, the isolated C₇₀ in the first round isolation process was not only in approximately 10 times the amount (72.6 mg) of the previous small scale experiments, but also in similar purity (99.0%) to the previous small scale experiments. Thus, the amount of C₇₀ isolated in a single purification cycle should be scalable to even greater levels if a greater amount of CTV1 is applied.

In this large-scale experiment, the total amount of the CTV1 that we recovered after chromatography and precipitation from toluene was 360 mg (72% recovery). Concentrating the CH₂Cl₂ phase obtained after dissociation of the hemicarceplex under reduced pressure allowed recycling of 96 mg of the hemicarceplex C₇₀⊙CTV1 (26% recovery, containing 64 mg of CTV1). Therefore, the mass loss of the CTV1 throughout the whole isolation process was approximately 15%. Because the dissociation of the hemicarceplex did not require competing guests, the recycled CTV1 could be used directly in a subsequent isolation cycle without the need for any specific treatment or purification process.

In the second round of isolation process, dissociation of the recycled hemicarceplex under similar conditions afforded C₇₀ in 92.6% purity (HPLC analysis). Notably, based on HPLC analysis, the C₇₀ isolated using this method was only negligibly contaminated with C₆₀ (0.05%); its major impurities were C₇₆ (6.00%) and C₇₈ (0.65%). Therefore, the CTV1 appears to also isolate C₇₆ and C₇₈ from the fullerene extract. Based on HPLC analysis of the commercial fullerene extract that we tested in this study, the ratio of C₇₆, C₇₈, and C₈₄ was approximately 1:1.1:1.6 (i.e., C₇₆ was a relatively minor component). Therefore, our CTV1 appears to be capable of kinetically differentiating these three buckyballs through the effective formation of the hemicarceplex C₇₆⊙CTV1 under the developed experimental conditions.

### Experiment 3

### Using CTV1 to Obtain a Mixture of C₇₀, C₇₆ and C₇₈ from High Fullerene Extract

The CTV1 (50 mg) and the high fullerene extract (50 mg; purchased from MER Corp.) were mixed in CHCl₂CHCl₂ (5 mL) and stirred at 40° C. for 18 h. The organic solvent was evaporated under reduced pressure and the residue was dissolved in CH₂Cl₂ (20 mL). After filtration, the solvent was evaporated under reduced pressure to afford a solid, which was purified through column chromatography [SiO₂ (8 g); eluent: CS₂ (50 mL) followed by CH₂Cl₂/hexane, 3:2 (400 mL), and CH₂Cl₂/MeOH, 49:1 (100 mL)].

The hemicarceplex mixtures (32.0 mg) were obtained and the above experiment was repeated three times and collected hemicarceplex mixture (105 mg) was then dissolved in toluene (5 mL) and heated at 30° C. for 6 h. The toluene solution was removed via pipette after centrifugation. Another charge of toluene (5 mL) was added to wash the white solid and then the mixture was centrifuged again. The white solid was recycled as the free CTV1.

The residue obtained after concentrating the combined toluene phases was suspended in CH₂Cl₂ (5 mL), in which the hemicarceplex mixtures are highly soluble, forming a red precipitate of fullerene mixtures, which were collected through centrifugation. The fullerene mixtures were then resuspended in CH₂Cl₂ (5 mL), centrifuged, separated from the solvent, and dried (4.8 mg). The purity was determined through HPLC analysis. The HPLC analysis results were listed in Table 8 below. From the HPLC result, it can be known that the mixture of C₇₀, C₇₆ and C₇₈ can be isolated from the mixture of the high fullerene extract.

TABLE 8
Analyzing the compositions of the purchased high fullerene extract
and the purity of the obtained black solid fullerene mixtures by HPLC

| | composition | | | | |
|---|---|---|---|---|---|
| | C₆₀ (%) | C₇₀ (%) | C₇₆ (%) | C₇₈ (%) | C₈₄ (%) |
| High fullerene extract | 4.2 | 6.8 | 12.9 | 14.0 | 65.8 |
| Obtained fullerene | 0 | 7.2 | 52.5 | 40.3 | 0 |
| mixtures | | | | | |

### Experiment 4

### Using CTV5 to Obtain Mixture of C₇₆ and C₇₈ from Mixture of C₇₀, C₇₆ and C₇₈

The CTV5 (1.5 mg) and the fullerene mixture [0.75 mg; the one contains only C₇₀, 076 and C₇₈ obtained from the Experiment 3] were mixed in CHCl₂CHCl₂ (0.4 mL) at 27° C. for 21 h. The organic solvent was evaporated under reduced pressure to afford a solid, which was purified through column chromatography [SiO₂ (0.4 g); eluent: CS₂ (2 mL) followed by EA/hexane, 3:7 in volume ratio (10 mL)]. The hemicarceplex mixture was then dissolved in toluene (0.8 mL) and heated at 50° C. for 40 h. The toluene solution was analyzed by HPLC. The HPLC analysis results were listed in Table 9 below. From the HPLC analysis result, it can be known that the mixture of C₇₆ and C₇₈ can be isolated from the mixture of C₇₀, C₇₆ and C₇₈.

TABLE 9
HPLC analysis result

| | composition | | |
|---|---|---|---|
| | C₇₀ (%) | C₇₆ (%) | C₇₈ (%) |
| Initial fullerene mixture | 7.2 | 52.5 | 40.3 |
| Isolated fullerene mixture | 3.9 | 44.8 | 51.3 |

In light of the foregoing, various CTV hosts can form complexes or hemicarceplexes with various fullerenes. The hemicarceplexes of fullerene⊙CTV can be used to isolate a fullerene or a fullerene mixture within a certain steric size range from a fullerene mixture, without using HPLC or recrystallization techniques. The most remarkable is that CTV1 can be used to isolate C₇₀ in high purity (≧99.0%) from a commercial fullerene extract.

The preparation of hemicarcerands that isolate C₇₀ and higher fullerenes suggests the possibility of not only isolating and stabilizing these novel molecules, their analogues, and derivatives but also applying them practically as useful photovoltaic materials by significantly increasing their solubility in less-polar solvents without covalently disrupting their unique pi-surfaces. Moreover, by elongating or shortening the linking spacers of the CTV hosts, selective trapping of fullerenes with various sizes and the possibility of using them as photovoltaic materials are allowed.

All the features disclosed in this specification (including any accompanying claims, abstract, and drawings) may be replaced by alternative features serving the same, equivalent or similar purpose, unless expressly stated otherwise. Thus, each feature disclosed is one example only of a generic series of equivalent or similar features.

1. A fullerene⊙CTV complex formed by trapping a fullerene guest or a derivative thereof in a cyclotriveratrylene-based molecular cage (abbreviated as CTV below) having a chemical structure below: 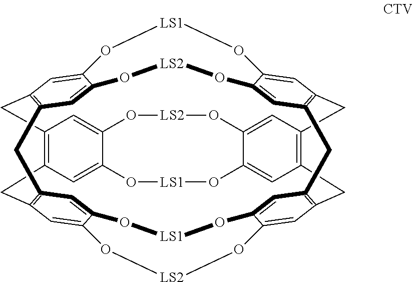
wherein LS1 and LS2 are first and second linking spacers.

2. The complex of claim 1, wherein at least three of the first and the second linking spacers are alkyl chains containing at least 10 carbons.

3. The complex of claim 2, wherein the cyclotriveratrylene-based molecular cage is

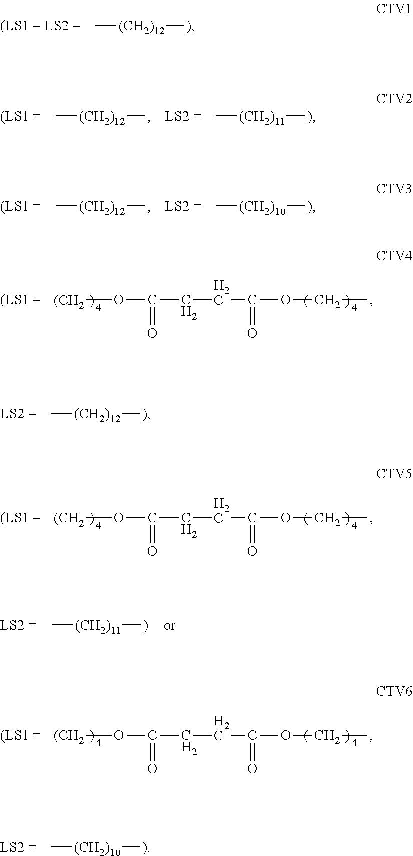

4. The complex of claim 3, wherein the complex is C₆₀⊙CTV1, C₇₀⊙CTV1, C₇₆⊙CTV1, C₇₈⊙CTV1, C₆₀⊙CTV2, C₇₀⊙CTV2, C₆₀⊙CTV3, Sc₃N@C₈₀⊙CTV4, C₆₀⊙CTV5, C₇₀⊙CTV5, C₇₆⊙CTV5 or C₇₈⊙CTV5, or C₆₀⊙CTV6.

5. The complex of claim 1, wherein at least one of the first and the second linking spacers containing a diester linkage.

6. The complex of claim 5, wherein the cyclotriveratrylene-based molecular cage is

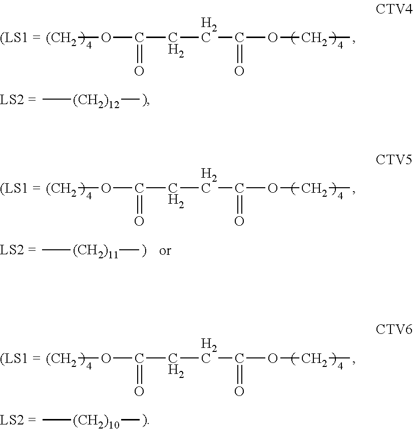

7. The complex of claim 6, wherein the complex is Sc₃N@C₈₀⊙CTV4, C₆₀⊙CTV5, C₇₀⊙CTV5, C₇₆⊙CTV5 or C₇₈⊙CTV5, or C₆₀⊙CTV6.

8. A method of forming a fullerene⊙CTV hemicarceplex, the method comprising:
mixing a fullerene or a derivative thereof, and a cyclotriveratrylene-based molecular cage (abbreviated as CTV below) in a solvent to form a mixture solution, wherein the CTV has a chemical structure shown below, and LS1 and LS2 are first and second linking spacers.

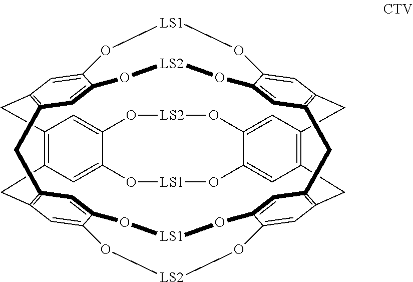

9. The method of claim 8, wherein at least three of the first and the second linking spacers are alkyl chains containing at least 10 carbons.

10. The method of claim 8, wherein at least one of the first and the second linking spacers containing a diester linkage.

11. The method of claim 8, wherein the solvent majorly contains CS₂, CH₂Cl₂, CHCl₃ or CHCl₂CHCl₂.

12. The method of claim 8, further comprising heating the mixture solution to form a fullerene⊙CTV hemicarceplex.

13. The method of claim 12, wherein the mixture solution is heated at a temperature above room temperature to 80° C.

14. A method of isolating at least a fullerene by using a fullerene⊙CTV hemicarceplex, the method comprising:
forming at least the fullerene⊙CTV hemicarceplex by mixing a mixture of fullerenes or derivatives thereof, and a cyclotriveratrylene-based molecular cage (abbreviated as CTV below) in a first solvent, wherein the first solvent has less tendency than the fullerenes to occupy an inner space of the cyclotriveratrylene-based molecular cage, and wherein the CTV has a chemical structure shown below, and LS1 and LS2 are first and second linking spacers; 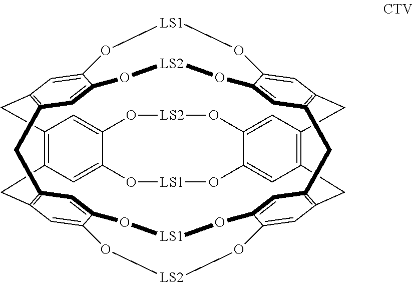
isolating the fullerene⊙CTV hemicarceplex by column chromatography and
dissociating the fullerene⊙CTV hemicarceplex in a second solvent, wherein the second solvent can dissolve the fullerene⊙CTV hemicarceplex and allow dissociating the fullerene⊙CTV hemicarceplex to release fullerene.

15. The method of claim 14, wherein at least three of the first and the second linking spacers are alkyl chains containing at least 10 carbons.

16. The method of claim 14, wherein at least one of the first and the second linking spacers containing a diester linkage.

17. The method of claim 14, wherein the first solvent majorly contains CS₂, CH₂Cl₂, CHCl₃, or CHCl₂CHCl₂.

18. The method of claim 14, wherein the second solvent majorly contains CS₂, CH₂Cl₂, CHCl₃, CHCl₂CHCl₂, benzene, toluene, or dichlorobenzene.
